SEQUENCE LISTING (22 sequences)

SEQ ID NO: 1 - DNA, 500 bp, Mycobacterium tuberculosis
catcgggtcaagcaccatgaccggtacatccgtcaggtcgtcgggcagcgagtccagatacggcaccggctggtgggtttgctcgtcgcgggcgacaccgacaaagccaacgtgcgcctccggcaaggcggcatgcgcctcgtcgaccatccccaaccccgcccgcaacacaggaaccagcaggggtggcttggttagccgcgacccgaccgtctcggccagcggcgtacggatcgggactggctcgcagggcgcatcgcgggtggcctcatagatcaacagcagcgtgagctcgcgcagcgctgcccggaagccggcgttgtcggtgcgttcgtcacgcagcgtggtcagtcgggccgcggccagtgggtggtcaacgacatggacctgcacggcgttgaaccctatataacaatcgtggctcggtcccctaaaagggggctgatacgggtgcgtccatccgcgcgaccggtcaaccccgtccatatactcccggcatg

SEQ ID NO: 2 - DNA, 500 bp, Mycobacterium bovis; identical to SEQ ID NO: 1

SEQ ID NO: 3 - DNA, 500 bp, Mycobacterium avium
gccggtggccagcatcgggtccagcaccatcaccgacctgccggccagcttgtcgggcagcgccgccaaatacgggaccggccggtgggtttgctcgtcgcgggcgacgccgacgaagcccacctcggcctcgggcagcgcggcctgcgccgggtcgaccatgcccagcccggcgcgcagcaccggaaccagcagcggcggattgaccagtcgggtcccggcggccgcggccaccggggtgcggatccggaccgacttgcgcggcgcgtcgcggctggcctcgtagaccagcatcagcgtcagatcgcgcagcgcggcccgaaatccggcggtgtcggtgcgttcgtcgcgcagcaccgtcagccgggccgcggccaacgggtggtcgatcacgcacacgtccatctggtcgagggtatataacgatcgggcaaagccccgctgacacgcttgcccgccggccggaaacgccttaccgccgttcgtatactccgggcgtg

SEQ ID NO: 4 - DNA, 500 bp, Mycobacterium marinum
cgccggtggccagcatcgggtccagcaccattacgggtagcccggacaagtcgtcgggcagcgattcaagatatgggacgggctggtgggtctgctcattgcgggcgataccgacaaagccgacccgcgcctccggcagcgccgcatgcgcttcgtcgaccatgcccagtccggcgcgcagcaccggaaccagaagcggggggttggccagtcttaggcctgtcgtggccgcaagcggtgtacggatagcgacggattcggtgggcgctgcgcgggtcgcctcatagaccagtaccagtgtcagctcacgcaaggccttgcggaaagcagcgtttccggtgcgttcgtcacgcagcgcggtcaggcgggccgccgccagcgggtgatcaatgacgtggacttccacatgggtgaccctatataacaatcggattcaagccgctgacacgctccccctcctcgcggcgccgaggccgagccgcccatatactccgggcgtg

SEQ ID NO: 5 - protein, 43 aa, Mycobacterium tuberculosis
MLRGIQALSRPLTRVYRALAVIGVLAASLLASWVGAVPQVGLA

SEQ ID NO: 6 - protein, 43 aa, Mycobacterium bovis; identical to SEQ ID NO: 5

SEQ ID NO: 7 - protein, 42 aa, Mycobacterium avium
MSRENRSRRRLIGGAYRSLRLLGAVAAVALAASPLTPRTSLA

SEQ ID NO: 8 - protein, 40 aa, Mycobacterium marinum
MCGLKQRFTSTFRALAVLGAVAVSLPAHGSDAPPRIDLTA

SEQ ID NO: 9 - DNA, 900 bp, Mycobacterium tuberculosis
  CDS (1)..(900)
atgctccgcggaatccaggctctcagccggcccctgaccagggtataccgtgccttggcggtgatcggtgtcctggcagcatcgttgctggcctcatgggtcggcgctgtcccacaagtgggtctggcagcgagtgccctgccgaccttcgcgcacgtggtcatcgtggtggaggagaaccgctcgcaggccgccatcatcggtaacaagtcggctcccttcatcaattcgctggccgccaacggcgcgatgatggcccaggcgttcgccgaaacacacccgagcgaaccgaactacctggcactgttcgctggcaacacattcgggttgacgaagaacacctgccccgtcaacggcggcgcgctgcccaacctgggttctgagttgctcagcgccggttacacattcatggggttcgccgaagacttgcctgcggtcggctccacggtgtgcagtgcgggcaaatacgcacgcaaacacgtgccgtgggtcaacttcagtaacgtgccgacgacactgtcggtgccgttttcggcatttccgaagccgcagaattaccccggcctgccgacggtgtcgtttgtcatccctaacgccgacaacgacatgcacgacggctcgatcgcccaaggcgacgcctggctgaaccgccacctgtcggcatatgccaactgggccaagacaaacaacagcctgctcgttgtgacctgggacgaagacgacggcagcagccgcaatcagatcccgacggtgttctacggcgcgcacgtgcggcccggaacttacaacgagaccatcagccactacaacgtgctgtccacattggagcagatctacggactgcccaagacgggttatgcgaccaatgctccgccaataaccgatatttggggcgactag

SEQ ID NO: 10 - protein, 299 aa, Mycobacterium tuberculosis
MLRGIQALSRPLTRVYRALAVIGVLAASLLASWVGAVPQVGLAASALPTFAHVVIVVEENRSQAAIIGNKSAPFINSLAANGAMMAQAFAETHPSEPNYLALFAGNTFGLTKNTCPVNGGALPNLGSELLSAGYTFMGFAEDLPAVGSTVCSAGKYARKHVPWVNFSNVPTTLSVPFSAFPKPQNYPGLPTVSFVIPNADNDMHDGSIAQGDAWLNRHLSAYANWAKTNNSLLVVTWDEDDGSSRNQIPTVFYGAHVRPGTYNETISHYNVLSTLEQIYGLPKTGYATNAPPITDIWGD

SEQ ID NO: 11 - DNA, 900 bp, Mycobacterium bovis
  CDS (1)..(900)
atgctccgcggaatccaggctctcagccggcccctgaccagggtataccgtgccttggcggtgatcggtgtcctggcagcatcgttgctggcctcatgggtcggcgctgtcccacaagtgggtctggcagcgagtgccctgccgaccttcgcgcacgtggtcatcgtggtggaggagaaccgctcgcaggccgccatcatcggtaacaagtcggctcccttcatcaattcgctggccgccaacggcgcgatgatggcccaggcgttcgccgaaacacacccgagcgaaccgaactacctggcactgttcgctggcaacacattcgggttgacgaagaacacctgccccgtcaacggcggcgcgctgcccaacctgggttctgagttgctcagcgccggttacacattcatggggttcgccgaagacttgcctgcggtcggctccacggtgtgcagtgcgggcaaatacgcacgcaaacacgtgccgtgggtcaacttcagtaacgtgccggcgacactgtcggtgccgttttcggcatttccgaagccgcagaattaccccggcctgccgacggtgtcgtttgtcatccctaacgccgacaacgacatgcacgacggctcgatcgcccaaggcgacgcctggctgaaccgccacctgtcggcatatgccaactgggccaagacaaacaacagcctgctcgttgtgacctgggacgaagacgacggcagcagccgcaatcagatcccgacggtgttctacggcgcgcacgtgcggcccggaacttacaacgagaccatcagccactacaacgtgctgtccacattggagcagatctacggactgcccaagacgggttatgcgaccaatgctccgccaataaccgatatttggggcgactag

SEQ ID NO: 12 - protein, 299 aa, Mycobacterium bovis
MLRGIQALSRPLTRVYRALAVIGVLAASLLASWVGAVPQVGLAASALPTFAHVVIVVEENRSQAAIIGNKSAPFINSLAANGAMMAQAFAETHPSEPNYLALFAGNTFGLTKNTCPVNGGALPNLGSELLSAGYTFMGFAEDLPAVGSTVCSAGKYARKHVPWVNFSNVPATLSVPFSAFPKPQNYPGLPTVSFVIPNADNDMHDGSIAQGDAWLNRHLSAYANWAKTNNSLLVVTWDEDDGSSRNQIPTVFYGAHVRPGTYNETISHYNVLSTLEQIYGLPKTGYATNAPPITDIWGD

SEQ ID NO: 13 - DNA, 903 bp, Mycobacterium avium
  CDS (1)..(903)
gtgtcgcgcgaaaatcgaagtcgcagaaggctgatcggcggcgcataccgaagcctgcggctgctcggcgccgtggccgcggtggcgctggcggccagcccgttgacaccgcgcaccagccttgcggcagcggccattccgcaaccgtcgcacatcgtgatcgtggtggaggaaaaccgttccgagagcggcatcatcggcaacaagtcggcgcccttcatcaccgcgctggccgcgtccggcgccaacatgacccagtcgttcgccgaaacccaccccagcgagcccaattacctggcgctgttcgccggcaacacgttcggggtgaccaaggacctgtgcccggtcaacgccggcgccgcacccaacctggggtccgaattgctcgccgccggttacacattcgccggctacgccgagggcctgccgtccccgggctcaccggtgtgcagtgcgggcaagtacgcgcgaaaacatgtgccgtgggccaacttcaccaacgtgccggcggcgagctcgctgccgttctcggcgttcccgatgggcaactacgccagcctgccgacggtgtcgttcgtcatcccgaacaacgacaacaacatgcacgacggctcgatcgcgcaggccgacgcctggctgaaccggcagctgtccggctacgccaattgggcgctggccaacaacagcctgctgatcgtcaccttcgacgaggacgacaactccaacgtcggagccagccgcaaccagatccccacggtgttctacggcgcccacgtccgccccggcaactacgccgagcagatcaaccactacaacgtgcttgccaccctcgagcagatgtacgggctgcccaagacgggctatgccgccggcgccgcccccatcaccgacatctggggctga

SEQ ID NO: 14 - protein, 300 aa, Mycobacterium avium
MSRENRSRRRLIGGAYRSLRLLGAVAAVALAASPLTPRTSLAAAAIPQPSHIVIVVEENRSESGIIGNKSAPFITALAASGANMTQSFAETHPSEPNYLALFAGNTFGVTKDLCPVNAGAAPNLGSELLAAGYTFAGYAEGLPSPGSPVCSAGKYARKHVPWANFTNVPAASSLPFSAFPMGNYASLPTVSFVIPNNDNNMHDGSIAQADAWLNRQLSGYANWALANNSLLIVTFDEDDNSNVGASRNQIPTVFYGAHVRPGNYAEQINHYNVLATLEQMYGLPKTGYAAGAAPITDIWG

SEQ ID NO: 15 - DNA, 888 bp, Mycobacterium marinum
  CDS (1)..(888)
gtgtgtggcctgaaacagcgttttaccagtacatttcgagctctggcggtactcggcgcggtggcggtatccctaccggcccacggtagcgacgctcccccgcgtatcgacctgaccgccactgcgttgccggcgttctcacatgtggtggtcgtggtggaggagaaccattcgcaggccaacatcattggcaacaaggcggccccgttcatcaatgcgctggccgccaatggcgcgatgatgtcgcagtcgttcgccgaaacgcaccccagcgaacccaactacctggccttgttcgccggtaccaccttcggcttgaagaagaacacgtgtccggtcaatgcgggcagcacgcccaacctggcttcggagttgctcgccgcgggccacacgttcgtaggtttcgccgagagcctgcccgaagtcggttcgacggtctgcagcgccggaaagtacgggcgcaagcatgcgccttgggtgaacttcagcaatgttccggccacgctgtcgatgcccttctccgcgtttccgacgccggcggactacgccaggctgcccacggtgtccttcgtcatccccaacggggataacaacatgcacgacggcaccatcgcggcagctgacgagtggttgaaccgtcaactgtcgccgtacgccaactgggcccgatccaacaacagcctgctgatcgtgacgtgggatgaggacgacggcggcagccgcaaccagattcccacggtgttctacggcgcacacgtacggccgggcacttacaaccagaccatcagccactacaacgtgctttccacgctggagcagatgtacggcttgcccaagacgggtttcgcggcgaacgccccggtcatcgctgatatctggggcggctaa

SEQ ID NO: 16 - protein, 295 aa, Mycobacterium marinum
MCGLKQRFTSTFRALAVLGAVAVSLPAHGSDAPPRIDLTATALPAFSHVVVVVEENHSQANIIGNKAAPFINALAANGAMMSQSFAETHPSEPNYLALFAGTTFGLKKNTCPVNAGSTPNLASELLAAGHTFVGFAESLPEVGSTVCSAGKYGRKHAPWVNFSNVPATLSMPFSAFPTPADYARLPTVSFVIPNGDNNMHDGTIAAADEWLNRQLSPYANWARSNNSLLIVTWDEDDGGSRNQIPTVFYGAHVRPGTYNQTISHYNVLSTLEQMYGLPKTGFAANAPVIADIWGG

SEQ ID NO: 17 - DNA, 1291 bp, Penicillium chrysogenum
  CDS (1)..(213)
  intro (217)..(244)
  CDS (245)..(1288)
atgctcaccaaacaaacccttctcgcgttcgtcggggccctcgccctcgccacgggtacaactaccactgaagagaccccaactcaggctgagattgatgcagcacgtgctacggccctgccttactctcctgtgtcaaacgtaaagggtttggcctttgatcgtttcgtgaacatctggctcgagaacacagtaggtttcccgttgaatatataacaatgaccacgcgctcacccctttgtaggactttgaacccgctgctttagacgagaacctgtccaagctggccaaggagggtatcctcctgaccaactactttgccatctctcacccctcgcagcccaactactgtgcttccgccgggggtgacacattcggcatggataatgacgacttcctacaaatcccttcgaatgtctcaactattgccgatctctttgatactaagcacatctcttggggtgaataccaagaagacatgccctatgctggctaccaaggcaaacggtatcccctcagcggtccgaaccagtacgtgcgcaagcacaacccgctggttttgtttaactcggttaccgacgacgccgtgcgcccgcgccaaatcaagaatttcaccactttctacgacgatctgaagcaccacagccttccccaacacatgttcatcacaccgaacatgaccaatgacgcccacgacacgaacatcactgtggccggtaactgggtcgatcgcttcctgtctcctctactgaagaacgagtacttcaccaaggacagcctagtgctactcacctttgacgagggagacacctactcctaccccaaccgggtcttcagcttccttgttggaggtgctatcccagagcacctgaaggggaccactgacgacactttctacacccactactcaattgtcgcttccctgtctgctaactggggtttaccctcgcttggtcgctgggattgtggcgccaacctgctgaagatggtcgctgacaagaccggctatgtcaactgggaagttgataccagcaatgtctacctcaacgagacttaccctggacctatgtctaccgacaactattcctctaagtgggccgttcctgccaccaagggcaaatgctctgctggccatggcattgctgaggtcgtgaagaatacctaccacgggcttcaacccacctacgactatgccagccctgtaccgtatgacgtgaccagtggaaacaacgtcggcatcaagtaccaccgcactctggtatgtatcctttcatgttcttccctttcatga

SEQ ID NO: 18 - protein, 71 aa, Penicillium chrysogenum
MLTKQTLLAFVGALALATGTTTTEETPTQAEIDAARATALPYSPVSNVKGLAFDRFVNIWLENTVGFPLNI

SEQ ID NO: 19 - protein, 348 aa, Penicillium chrysogenum
DFEPAALDENLSKLAKEGILLTNYFAISHPSQPNYCASAGGDTFGMDNDDFLQIPSNVSTIADLFDTKHISWGEYQEDMPYAGYQGKRYPLSGPNQYVRKHNPLVLFNSVTDDAVRPRQIKNFTTFYDDLKHHSLPQHMFITPNMTNDAHDTNITVAGNWVDRFLSPLLKNEYFTKDSLVLLTFDEGDTYSYPNRVFSFLVGGAIPEHLKGTTDDTFYTHYSIVASLSANWGLPSLGRWDCGANLLKMVADKTGYVNWEVDTSNVYLNETYPGPMSTDNYSSKWAVPATKGKCSAGHGIAEVVKNTYHGLQPTYDYASPVPYDVTSGNNVGIKYHRTLVCILSCSSLS

SEQ ID NO: 20 - DNA, 1457 bp, Aspergillus fumigatus
  CDS (1)..(186)
  Intron (187)..(239)
  CDS (240)..(301)
  Intron (302)..(361)
  CDS (362)..(1454)
atgaagccttccgtcgcgactttgcttgccactgtctctctggtctatgctcagactgctactgagaaggagccttcgctgtctgcgatagaatctgcagcagcctccatccagccttactctcccgtttcgaacgttgagggtgttgcatttaatcgcttcttccaagtgtggcttgagaatattgtatgtgatcaccttaccaatcagagaatcttgttcaaagctgacctcagaaggattacgaggatgctgcggcggatgagaacatgaaatggctggcctcgcaagggatcctgctgtaagacctcatatcggccatctgctcacgatttgaacttcgaactaatttactccacagcaccaatttctatgcagtcacgcatccttcagagccaaactactgcgctgctgttggaggcgacacatttggcatggacaatgacaactttaaccagattcctgccaatgtttctactgtcgctgatctcctggacaccaaaaacattgcttggggagagtatcaggagcacttaccttatcccggattccaaggtttcaactattccaaccaggagacttatgtcaatgactatgtgcgcaagcataacccactggtcttgtatgactctgtcaccaagaacagcactcgtttgcgccagatcaagaactttaccagcttcgaggacgacctggccaacaagaaacttcctcaatgggcatttatcactccaaacatgaccaacgacgctcatgacaccaacattactttcggagccaaatgggagcgaagctggattgcgcccttgctcaacaactcatacttcatgaatgataccctaatcctacttacctttgatgaggatggcacttattccaagagcaacaagatcttcagtgttcttctcggtggtgccattcccgatgagctgaagggtactcaggacgatacgttctatacccactactcagtgattgcgtccgtgtccgcgaactggggccttccttcgttgggaaggtgggattgtggtgcgaacattcttgagattgtggcaaacaagacgggatatgtcaactacgacgttgacacaaccaatctccgcctcaacgagacctaccccggtcccatgtcagcgggcgaatactcgaaatactcccctgtctggccgaatgccttgacccgtggtgactgctctgctggccatggcattttggacattgtcaaggagacctacgccaacacggagccaacatacaactattcgagccccttcccatatgacactgcgagcaactacaacaccaaggtgactgccaccaaaaagaatgtcaccggtacacatagaagttcttcttcctcctctccgtcagctagctccaacgccgctgtttctgctgtcgctcctgcagccggtgtctctggtctcctcttgggactcgctctaaacctgctttaa

SEQ ID NO: 21 - protein, 447 aa, Aspergillus fumigatus
MKPSVATLLATVSLVYAQTATEKEPSLSAIESAAASIQPYSPVSNVEGVAFNRFFQVWLENIDYEDAAADENMKWLASQGILLTNFYAVTHPSEPNYCAAVGGDTFGMDNDNFNQIPANVSTVADLLDTKNIAWGEYQEHLPYPGFQGFNYSNQETYVNDYVRKHNPLVLYDSVTKNSTRLRQIKNFTSFEDDLANKKLPQWAFITPNMTNDAHDTNITFGAKWERSWIAPLLNNSYFMNDTLILLTFDEDGTYSKSNKIFSVLLGGAIPDELKGTQDDTFYTHYSVIASVSANWGLPSLGRWDCGANILEIVANKTGYVNYDVDTTNLRLNETYPGPMSAGEYSKYSPVWPNALTRGDCSAGHGILDIVKETYANTEPTYNYSSPFPYDTASNYNTKVTATKKNVTGTHRSSSSSSPSASSNAAVSAVAPAAGVSGLLLGLALNLL

SEQ ID NO: 22 - protein, 8 aa, Mycobacterium tuberculosis
NDMHDGSI

(19) United States
(12) Patent Application Publication
(10) Pub. No.: US 2006/0172301 A1
(43) Pub. Date: Aug. 3, 2006

(54) Secreted acid phosphatse (sapm) is present only in pathogenic mycobacteria and expressed selectively at phagosomal ph

(76) Inventors: Jun Liu, Toronto (CA); Mazen T. Saleh, Brunswick, CA (US); David Alexander, Quebec (CA)

Correspondence Address: HARNESS, DICKEY & PIERCE, P.L.C., P.O. BOX 828, BLOOMFIELD HILLS, MI, 48303, US

(21) Appl. No.: 10/530,983

(22) PCT Filed: Oct. 9, 2003
(86) PCT No.: PCT/CA03/01554

§ 371 Date: Apr. 11, 2005

(30) Foreign Application Priority Data
Oct. 9, 2002 (US) 60416957

(51) Int. Cl. C12Q 1/68; G01N 33/554; C07H 21/04; C12P 21/06; C12N 9/16; C12N 15/74; G01N 33/569

(52) U.S. Cl. 435/6; 435/7.32; 435/69.1; 435/196; 435/252.3; 435/471; 530/388.26; 536/23.2

(57) ABSTRACT

The invention relates to an isolated DNA construct, comprising a mycobacterium secreted acid phosphatase promoter or promoter fragment which is inducible by low pH. This invention further relates to diagnostic methods and vaccines for treatment or prophylaxis of pathogenic mycobacterial disease or infection. This invention also provides for methods of screening for compounds capable of modulating the activity, production or secretion of a mycobacterium secreted acid phosphatase.

### FIELD OF INVENTION

This invention relates to diagnostic methods and vaccines for pathogenic mycobacterial or pathogenic fungal disease or infection.

### BACKGROUND

Pathogenic mycobacteria including Mycobacterium tuberculosis reside within the phagosome of the host macrophage, a harsh environment that is detrimental to most microbes. It is evident that M. tuberculosis modifies this phagosomal compartment in order to enhance its own intracellular survival (Clemens and Horwitz, 1995). This includes alterations in Rab GTPase composition (Via et al., 1997; Clemens et al., 2000) and exclusion of the vacuolar proton ATPase (Sturgill et al., 1994). Consequently, the mycobacterial phagosome is arrested at an early stage of maturation and only mildly acidified to pH 6.1-6.5 (Xu et al., 1994; Sturgill et al, 1994; Clemens and Horwitz, 1995). The ability of pathogenic mycobacteria to persist in macrophages plays a crucial role in disease establishment and progression. However, the specific bacterial factors that undermine the host processes remain largely unknown. Acting on the assumption that genes contributing to bacterial pathogenesis are differentially expressed during infection, various approaches have been used to identify M. tuberculosis genes whose expression are preferentially upregulated in vivo (i.e., within macrophages) (reviewed in (Triccas and Gicquel, 2000) or under in vitro conditions that mimic the host environment encountered by the bacillus (e.g., low oxygen tension, acidic pH, and nutrient starvation) (Betts et al., 2002; Fisher et al, 2002). Although a number of candidate virulence genes have been described, many have no known biological function and their direct roles on intracellular survival of the bacillus remain obscure. The identification of mycobacterial proteins that contribute to the replication and survival of the bacteria is critical for understanding the pathogenesis and protective mechanisms of the disease.

Intracellular bacterial pathogens including M. tuberculosis and Salmonella enterica serovar Typhimurium encounter a wide variety of environmental conditions within the host that are not accessible to commensal species. The ability to respond to environmental cues, and adjust to harsh intracellular conditions allows these pathogens to survive. Environmental cues, including temperature, osmolarity, pH, oxygen tension, and nutrient availability, have been found to act as signals for the induction of specific subsets of bacterial genes, which contributes to enhanced survival or multiplication. Many of these genes have been identified in Salmonella, and some have been shown to play important roles in infection and/or pathogenesis (reviewed in (Lucas and Lee, 2000). Using a variety of strategies, a number of candidate virulence genes of M. tuberculosis have also been identified (Triccas and Gicquel, 2000). Yet direct evidence supporting a necessary role in virulence is only available for a few genes, e.g., isocitrate lyase (aceA) (McKinney et al., 2000), α-crystallin (acr) (Yuan et al., 1998), and the exported repetitive protein (erp) (Berthet et al., 1998).

It is well known that pathogenic mycobacteria actively modify the phagosomal pH. Phagosomes containing viable pathogenic M. tuberculosis or M. avium are only mildly acidified (pH 6-6.5), which is, at least partially, due to the paucity of proton ATPase (Sturgill et al., 1994). In contrast, nonpathogenic mycobacteria such as M. smegmatis and Mycobacterium gordonae, or dead pathogenic mycobacteria, do not block phagosomal acidification and the pH of phagosomes containing these bacteria is acidified to values similar to the pH of late endosomes and lysosomes (pH ≈6) (Kuehnel et al., 2001). These data suggest that productions of factors found only in pathogenic strains are important for inhibition of phagosomal acidification and intracellular survival. Acidification may be a signal to induce expression of genes needed to alter phagosomal maturation. SapM, the secreted acid phosphatase of M. tuberculosis H37Rv, was identified recently (Saleh and Belisle, 2000).

### SUMMARY

This invention provides an isolated DNA sequence comprising a promoter or promoter fragment from the 5′ flanking region upstream of the start site of the coding region of a mycobacterial secreted acid phosphatase gene [SEQ ID NO:9], [SEQ ID NO:11], [SEQ ID NO:13], [SEQ ID NO:15] wherein the promoter or promoter fragment is sufficient to control expression of a nucleotide sequence of interest and is inducible under low-pH conditions. This invention also provides an isolated DNA sequence comprising a promoter or promoter fragment from the 5′ flanking region upstream of the start site of the coding region of a secreted acid phosphatase gene selected from the group consisting of Mycobacterium tuberculosis [SEQ ID NO:9], Mycobacterium bovis [SEQ ID NO:11], Mycobacterium avium [SEQ ID NO:13], or Mycobacterium marinum [SEQ ID NO:15].

The present invention relates to an isolated DNA sequence comprising a promoter or promoter fragment which hybridizes to [SEQ ID NO:1], [SEQ ID NO:2], [SEQ ID NO:3], [SEQ ID NO:4] under high stringency hybridization conditions and which is sufficient to control expression of a nucleotide sequence of interest and is inducible under low pH conditions. The present invention also relates to an expression vector comprising an isolated DNA sequence which comprises a promoter or promoter fragment from the 5′ flanking region upstream of the start site of the coding region of a mycobacterial secreted acid phosphatase gene [SEQ ID NO: 9], [SEQ ID NO:11], [SEQ ID NO:13], [SEQ ID NO:15] wherein the promoter or promoter fragment is sufficient to control expression of a nucleotide sequence of interest and is inducible under low-pH conditions.

The present invention also relates to an expression vector comprising an isolated DNA sequence which comprises a promoter or promoter fragment from the 5′ flanking region upstream of the start site of the coding region of a secreted acid phosphatase gene selected from the group consisting of Mycobacterium tuberculosis [SEQ ID NO:9], Mycobacterium bovis [SEQ ID NO:11], Mycobacterium avium [SEQ ID NO:13], or Mycobacterium marinum [SEQ ID NO:15].

The present invention also relates to an expression vector comprising an isolated DNA sequence which comprises a promoter or promoter fragment which hybridizes to [SEQ ID NO:1], [SEQ ID NO:2], [SEQ ID NO:3], [SEQ ID NO:4] under high stringency hybridization conditions and which is sufficient to control expression of a nucleotide sequence of interest and is inducible under low-pH conditions.

The invention further relates to a host cell transformed with an expression vector comprising an isolated DNA sequence which comprises a promoter or promoter fragment from the 5′ flanking region upstream of the start site of the coding region of a mycobacterial secreted acid phosphatase gene [SEQ ID NO:9], [SEQ ID NO:11], [SEQ ID NO:13], [SEQ ID NO:15] wherein the promoter or promoter fragment is sufficient to control expression of a nucleotide sequence of interest and is inducible under low-pH conditions. The invention further relates to a host cell transformed with an expression vector comprising an isolated DNA sequence which comprises a promoter or promoter fragment from the 5′ flanking region upstream of the start site of the coding region of a secreted acid phosphatase gene selected from the group consisting of Mycobacterium tuberculosis [SEQ ID NO:9], Mycobacterium bovis [SEQ ID NO:11], Mycobacterium avium [SEQ ID NO:13], or Mycobacterium marinum [SEQ ID NO:15].

The invention further relates to a host cell transformed with an expression vector comprising an isolated DNA sequence which comprises a promoter or promoter fragment which hybridizes to [SEQ ID NO: 1], [SEQ ID NO:2], [SEQ ID NO:3], [SEQ ID NO:4] under high stringency hybridization conditions and which is sufficient to control expression of a nucleotide sequence of interest and is inducible under low-pH conditions.

Another aspect of the invention is a transcription cassette comprising a sapM promoter or a promoter fragment [SEQ ID NO 1:], [SEQ ID NO:2], [SEQ ID NO:3], [SEQ ID NO:4], a nucleotide sequence of interest operably linked to the sapM promoter or promoter fragment [SEQ ID NO :1], [SEQ ID NO:2], [SEQ ID NO:3], [SEQ ID NO:41 and a transcriptional termination region. In one embodiment the transcription cassette further comprises a mycobacterial secreted acid phosphatase N-terminal signal sequence [SEQ ID NO:5], [SEQ ID NO:6], [SEQ ID NO:7], [SEQ ID NO:8] or a functional portion thereof. In another embodiment the transcription cassette further comprises the secreted acid phosphatase N-terminal signal sequence selected from the group consisting of Mycobacterium tuberculosis[SEQ ID NO:5], Mycobacterium bovis [SEQ ID NO:6], Mycobacterium avium [SEQ ID NO:7], or Mycobacterium marinum [SEQ ID NO:8].

In yet another aspect of the invention there is a method for the diagnosis of a pathogenic mycobacterial infection or a pathogenic fungal infection in a subject comprising obtaining a biological sample from a subject and analyzing the sample for the presence of antibodies specific to SapM [SEQ ID NO: 10], [SEQ ID NO:12], [SEQ ID NO:14], [SEQ ID NO:16] wherein detection of antibodies specific to SapM [SEQ ID NO:10], [SEQ ID NO:12], [SEQ ID NO:14], [SEQ ID NO:16] is indicative of the presence of the pathogenic mycobacterial infection or the pathogenic fungal infection. In one aspect of the invention there is a method for the diagnosis of a pathogenic mycobacterial infection or a pathogenic fungal infection in a subject comprising obtaining a nucleic acid sample from a subject and analyzing the sample for the presence of nucleic acid encoding sapM [SEQ ID NO:9], [SEQ ID NO:11], [SEQ ID NO:13], [SEQ ID NO:15] wherein detection of sapM [SEQ ID NO:9], [SEQ ID NO:!1], [SEQ ID NO:13], [SEQ ID NO:15] is indicative of the presence of the pathogenic mycobacterial infection of the pathogenic fungal infection. In one embodiment the method for the diagnosis of a pathogenic mycobacterial infection or a pathogenic fungal infection further comprises the step of quantifying the amount of sapM [SEQ ID NO: 9], [SEQ ID NO:11], [SEQ ID NO:13], [SEQ ID NO:15] detected. Another aspect of the invention is a method for the diagnosis of a pathogenic mycobacterial infection or a pathogenic fungal infection in a subject comprising obtaining a biological sample from a subject and analyzing the sample for the presence of SapM phosphatase activity wherein detection of SapM phosphatase activity is indicative of the presence of the pathogenic mycobacterial infection or the pathogenic fungal infection.

The invention also relates to a method of screening for a compound capable of modulating or regulating production of SapM [SEQ ID NO:10], [SEQ ID NO:12], [SEQ ID NO:14], [SEQ ID NO:16] comprising providing or employing a nucleic acid construct comprising the sapM promoter [SEQ ID NO:1], [SEQ ID NO:2], [SEQ ID NO:3], [SEQ ID NO:4] or a functional part thereof, the promoter or the functional part thereof being operably linked to a reporter gene capable of producing a detectable or a measurable signal, exposing the construct to a candidate or test compound and detecting or measuring the signal produced by the reporter gene, wherein a change in the signal produced in the presence of the candidate or test compound as compared to in the absence of the test compound indicates that the test compound is capable of modulating production of the mycobacterial secreted acid phosphatase. In one embodiment a plurality of compounds is tested.

The invention also relates to a method of screening for a compound capable of modulating or regulating SapM phosphatase activity comprising incubating a mixture comprising SapM [SEQ ID NO:10], [SEQ ID NO:12], [SEQ ID NO:14], [SEQ ID NO:16] a SapM substrate, and the compound to be tested, and measuring the phosphatase activity, wherein a change in activity in the presence of the test compound as compared to in the absence of the test compound indicates that the test compound is capable of modulating the activity of the secreted acid phosphatase. In one embodiment a plurality of compounds is tested.

The invention further relates to a method of screening for a compound capable of regulating or modulating SapM [SEQ ID NO:10], [SEQ ID NO:12], [SEQ ID NO:14], [SEQ ID NO:16] secretion comprising exposing mycobacterial cells to a compound to be tested, wherein the mycobacterial cells secrete SapM [SEQ ID NO:10], [SEQ ID NO:12], [SEQ ID NO:14], [SEQ ID NO:16] detecting the presence or activity of SapM [SEQ ID NO:10], [SEQ ID NO:12], [SEQ ID NO:14], [SEQ ID NO:16] secreted by the mycobacterial cells, and measuring the amount of the mycobacterial secreted acid phosphatase secreted by the mycobacterial cells, wherein a change in the secretion of the mycobacterial secreted acid phsophatase in the presence of the test compound as compared to in the absence of the test compound indicates that the test compound is capable of modulation of the secretion of the mycobacterial secreted phosphatase. In one embodiment a plurality of compounds is tested.

The invention also relates to a kit for the detection of a pathogenic mycobacterial disease or infection comprising a SapM protein [SEQ ID NO:10], [SEQ ID NO:12], [SEQ ID NO:14], [SEQ ID NO:16] or polypeptide thereof, at least one SapM antibody, and one or more reagents necessary for detection of SapM specific antibodies.

The invention also relates to a kit for the detection of a pathogenic mycobacterial disease or infection comprising an oligonucleotide comprising contiguous nucleotides from the nucleic acid sequence that is complementary to the sequence of [SEQ ID NO:1], [SEQ ID NO:2], [SEQ ID NO:3], [SEQ ID NO:4], [SEQ ID NO:9], [SEQ ID NO:11], [SEQ ID NO:13], [SEQ ID NO:15] and capable of specifically hybridizing to the complementary nucleotide sequence and reagents for hybridization of the oligonucleotide to a complementary nucleic acid sequence.

In another aspect of the invention there is an antibody which is capable of specifically binding to a SapM protein [SEQ ID NO:10], [SEQ ID NO:12], [SEQ ID NO:14], [SEQ ID NO:16] or a polypeptide fragment thereof [SEQ ID NO:22].

In yet another aspect of the invention there is a vaccine or immunogenic composition for treatment or prophylaxis of a mammal against challenge by a mycobacterium comprising an antibody which is capable of specifically binding to a SapM protein [SEQ ID NO:10], [SEQ ID NO:12], [SEQ ID NO:14], [SEQ ID NO:16] or a polypeptide fragment thereof [SEQ ID NO:22].

In a further aspect of the invention there is a vaccine or immunogenic composition for treatment or prophylaxis of a mammal against challenge by a mycobacterium comprising an isolated DNA sequence comprising a promoter or promoter fragment from the 5′ flanking region upstream of the start site of the coding region of a mycobacterial secreted acid phosphatase gene [SEQ ID NO:1], [SEQ ID NO:2], [SEQ ID NO:3], [SEQ ID NO:4] wherein the promoter or promoter fragment is sufficient to control expression of a nucleotide sequence of interest and is inducible under low-pH conditions. Another aspect of the invention relates to a vaccine or immunogenic composition for treatment or prophylaxis of a mammal against challenge by a mycobacterium comprising an isolated DNA sequence comprising a promoter or promoter fragment which hybridizes to [SEQ ID NO:1], [SEQ ID NO:2], [SEQ ID NO:3], [SEQ ID NO:4] under high stringency hybridization conditions and which is sufficient to control expression of a nucleotide sequence of interest and is inducible under low pH conditions.

The invention also relates to a vaccine or immunogenic composition for the treatment or prophylaxis of a mammal against challenge by a mycobacterium comprising a SapM protein [SEQ ID NO:10], [SEQ ID NO:12], [SEQ ID NO:14], [SEQ ID NO:16] or a polypeptide fragment thereof [SEQ ID NO:22]. Another aspect of the invention relates to a vaccine or immunogenic composition for treatment or prophylaxis of a mammal against challenge by a mycobacterium comprising an isolated DNA sequence or sequence fragment which hybridizes to [SEQ ID NO:9], [SEQ ID NO:11], [SEQ ID NO:13], [SEQ ID NO:15] under high stringency hybridization conditions and which allows for expression of a SapM protein [SEQ ID NO:10], [SEQ ID NO:12], [SEQ ID NO:14], [SEQ ID NO:16] or a polypeptide fragment thereof [SEQ ID NO:22]. The invention further relates to an antigenic composition useful for the detection of a pathogenic mycobacterial disease or infection in a subject comprising a SapM polypeptide and substantially free from other proteins or glycoproteins with which it is natively admixed in a culture of pathogenic mycobacteria.

### DETAILED DESCRIPTION OF THE DRAWINGS

Preferred embodiments of the invention will be described in relation to the drawings in which:

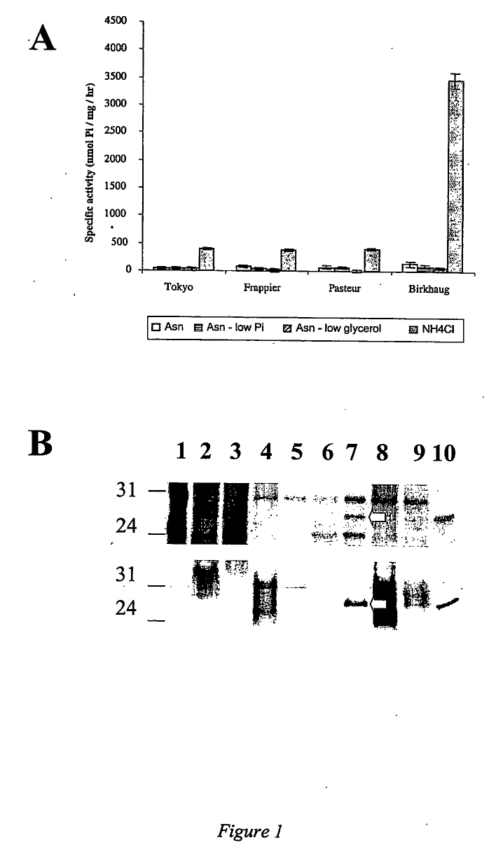

FIG. 1. (A) Acid phosphatase activity in the culture supernatant of M. bovis BCG in response to nutrient stress. BCG strains, in triplicate, were grown to early stationary phase in standard, asparagine-containing Sauton medium, low phosphate Sauton, low glycerol Sauton, or ammonium-containing Sauton, and the acid phosphatase activity in the culture supernatants were determined as described under “Experimental Procedures”. (B) SapM expression in M. bovis BCG grown in Sauton media. Equal amounts of protein (6 μg) were loaded in each lane and visualized by silver staining (top panel) or by Western blotting with SapM antisera (lower panel). Lanes 2, 4, 6, and 8 are from culture supernatant of BCG-Frappier, -Pasteur, -Birkhaug, and -Japan grown in normal Sauton medium, respectively. Lanes 3, 5, 7, and 9 are the corresponding strains grown in ammonium-containing Sauton medium. Lane 10 is partially purified SapM from mc²-155/pSAP, and lane 1 is molecular weight marker.

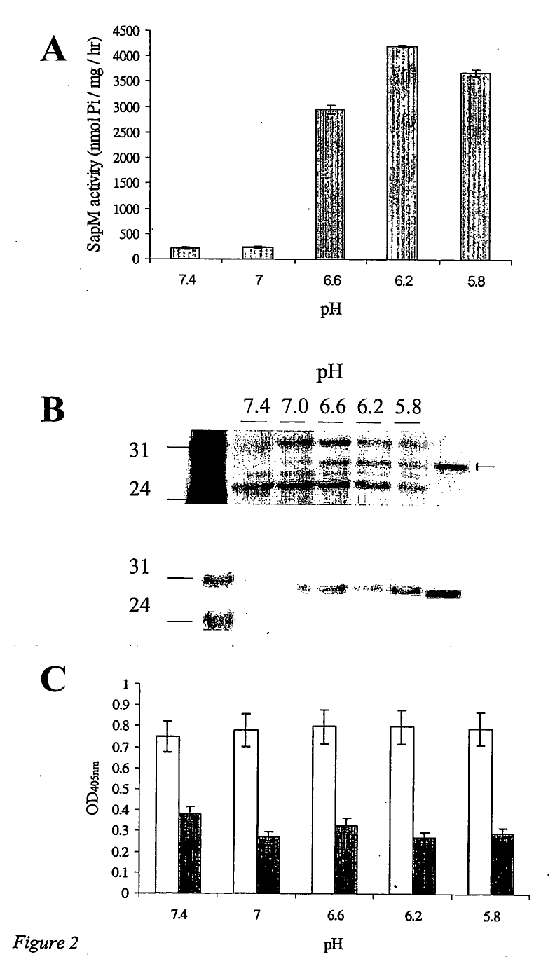

FIG. 2. pH-dependent expression of SapM in M. bovis BCG-Birkhaug. (A) Acid phosphatase activity determined in CFP fractions of BCG-Birkhaug cultures grown in Sauton medium in which the pH was adjusted to indicated levels by appropriate buffers. Results are from triplicate cultures. (B) The same CFP fractions as in panel (A) were subjected to SDS-PAGE analysis. Equal amounts of protein (7 μg) were loaded in each lane and visualized by silver staining (top panel) or by Western blotting with SapM antisera (lower panel). Partially purified SapM, indicated by the arrow, was included as a control. (C) Cell surface-associated acid phosphatase (open bars) and alkaline phosphatase (filled bars) activity from BCG-Birkhaug. The activities are detected from equal numbers of cell as described under “Experimental Procedures”.

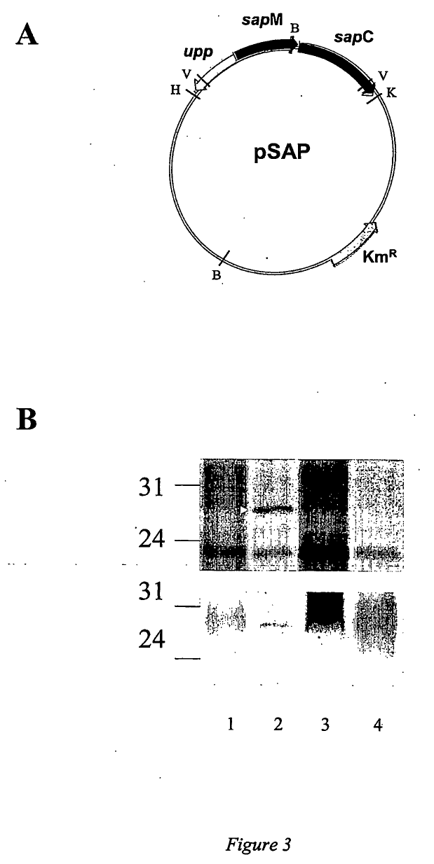

FIG. 3. Cloning and expression of M. tuberculosis sapM in M. smegmatis. (A) Construction of pSAP. A 3 kb NheI fragment of the M. tuberculosis genome (BAC403) was ligated to XbaI-linearized pMD31. The resulting plasmid, pSAP, contains upp, sapM (Rv3310) and sapC (Rv3311). Restriction enzyme sites: B Bgl II, H HindIII, K KpnI, V EcoRV, and X XbaI. (B) Expression of sapM in M. smegmatis . Cells of M. smegmatis were grown in normal asparagine-containing Sauton (lanes 1 and 3) or ammonium-containing Sauton media (lanes 2 and 4), and the CFP fractions were subjected to SDS-PAGE and visualized by silver staining (top panel) or by Western blotting with SapM antisera (lower panel). Lanes 1, 2: mc²-155/pSAP, lanes 3, 4: mc²-155/pMD31. Arrow indicates SapM.

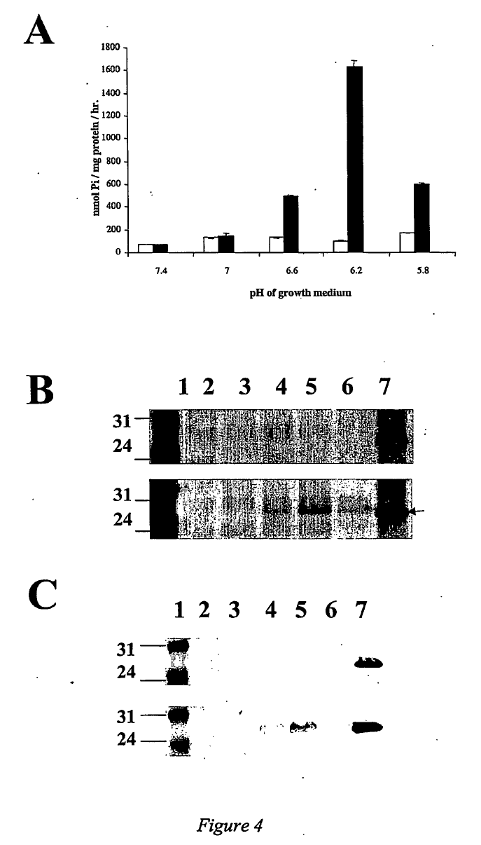

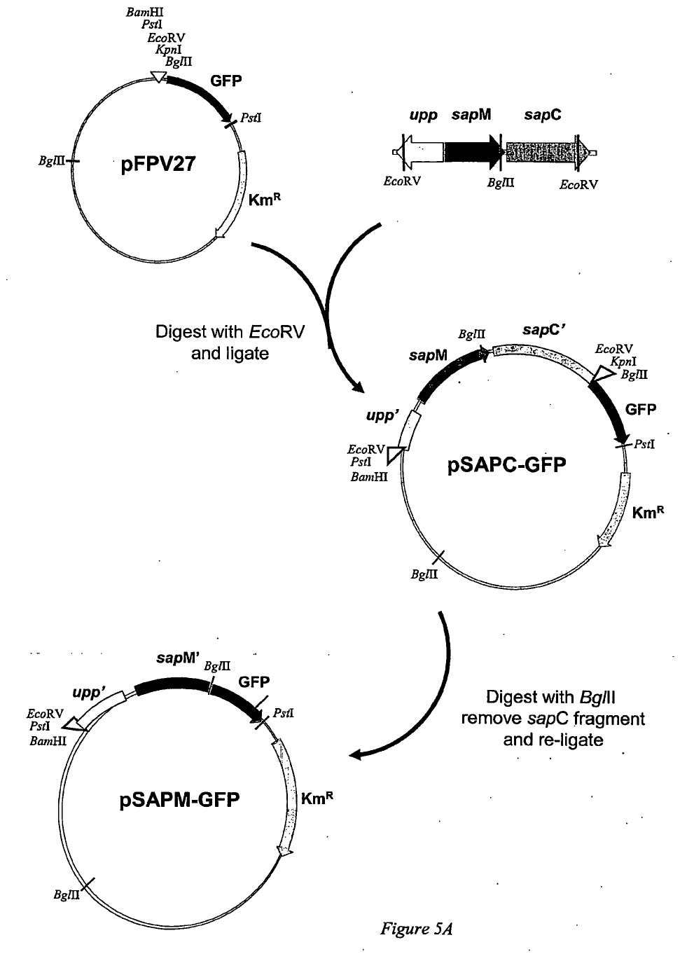

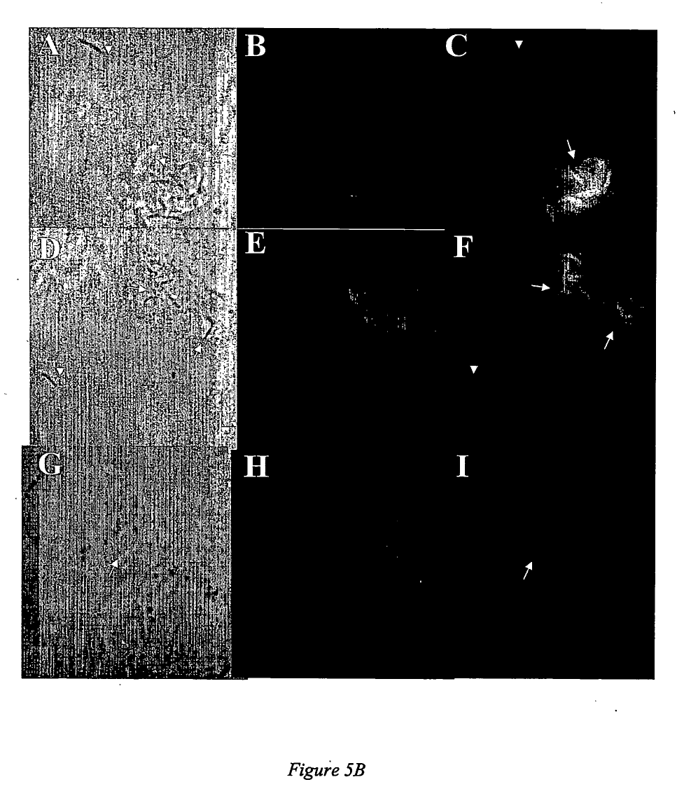

FIG. 4. pH-induced expression of M. tuberculosis sapM. (A) M. smegmatis recombinant strain mc²-155/pSAP (filled bars) and control strain mc 2-155/pMD31 (open bars) were grown in Sauton media adjusted to the indicated pH and the acid phosphatase activity in the CFP fractions were determined. (B & C) The same CFP fractions from panel A were subjected to SDS-PAGE analysis and visualized by (B) silver staining or (C) Western blotting. The top panel is from mc²-155/pMD31 and the lower panel from mc²-155/pSAP. Partially purified SapM, indicated by arrow, was included as a control.

FIG. 5. (A) Construction of GFP-fusion vectors. A 2.5 kb EcoRV fragment of pSAP was ligated to EcoRV-linearized GFP-fusion vector pFPV27. The resulting pSAPC-GFP contains the entire sapM gene and a truncated sapC gene transcriptionaly fused to gfp. Vector pSAPC-GFP was then cut with BglII. The 3.8 kb and 2.5 kb fragments, comprising sapM, gfp and the vector backbone, were re-ligated. The resulting pSAPM-GFP contains a truncated sapM gene transcriptionaly fused to gfp. (B) J774 cells were infected with M. marinum bearing pSAPM-GFP (panels A-C), pSAPC-GFP (panels D-F), or cloning vector pFPV27 (panels G-H) and examined by microscope. M. marinum cells were grown in Sauton medium (pH 7.4) and J774 cells were infected with the bacteria for 24 hrs before analysis. Images were collected for each view in the DIC mode (panels A, D, and G), in the red fluorescence channel for Texas-red phalloidin (panels B, E, and H), and in the green fluorescence channel (panels C, F, and I). The intracellular bacteria are designated by arrows and extracellular bacteria are designated by arrowheads.

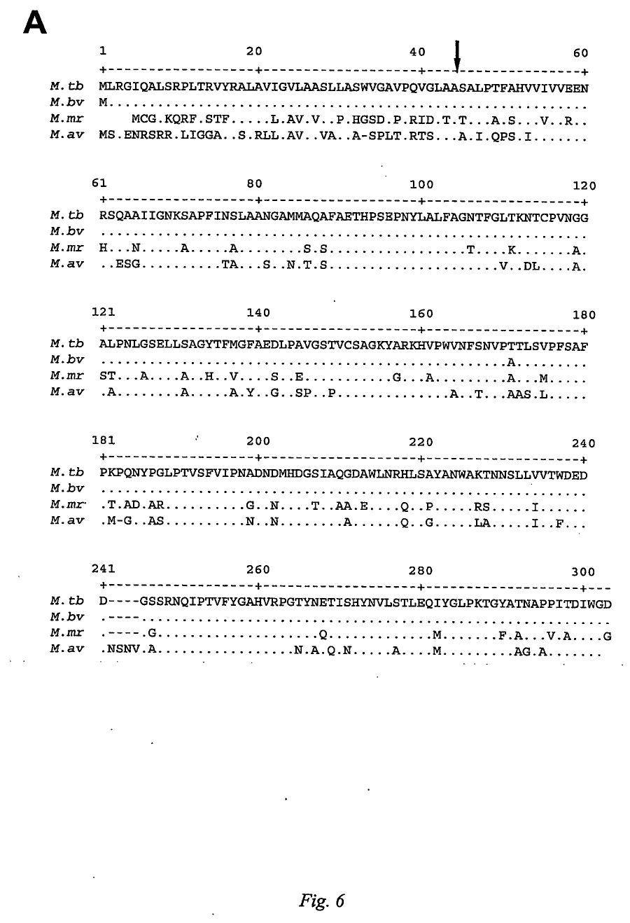

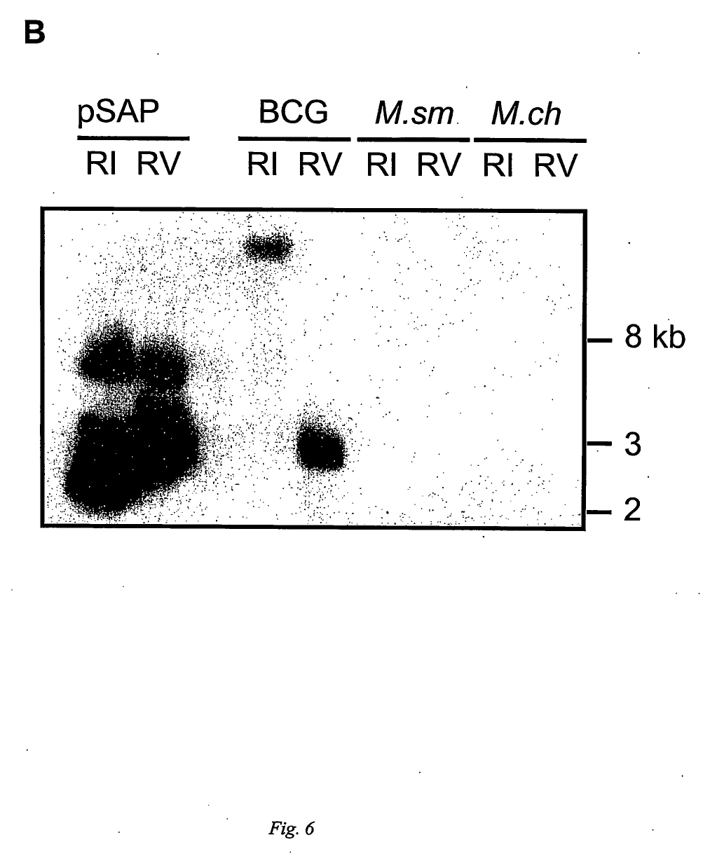

FIG. 6. (A) Alignment of SapM homologs found in the genome database of M. tuberculosis, M. bovis, M. avium and M. marinum. Arrow indicates putative signal sequence cleavage site. (B) Southern bolt analysis. Plasmid pSAP, and chromosomal DNA isolated from in M. bovis BCG, M. smegmatis, and M. chelonae were digested with EcoRI or EcoRV, separated on an agarose gel, transferred to a nylon membrane, and hybridized with a radiolabeled sapM probe.

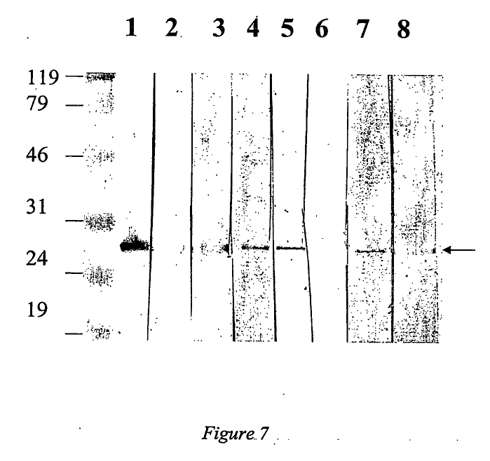

FIG. 7. Reactivity of human sera with partially purified SapM from mc²-155/pSAP. Partially purified SapM was run on SDS-PAGE and transferred to nitrocellulose membranes. Individual lanes were cut and probed with a 1:150 dilution of human sera. Lanes: 1, rabbit SapM antisera generated against a synthetic peptide as described under “Experimental Procedures”; 2, sera from a healthy individual with a positive PPD test; 3 and 6, healthy individuals with unknown PPD reactivity; 4, patient with lymph node tuberculosis who has been on chemotherapy for 4 months; 5, patient with evident M. avium in sputum; 7, healthy individual who was vaccinated with BCG in 1977; 8, sera from a healthy individual who was vaccinated with BCG in 1953.

### DETAILED DESCRIPTION

The present invention will now be described more fully with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

The term “antibody” as used herein, is meant to include both the native antibody, and biologically active derivatives of antibodies, such as, for example, Fab′, F(ab′).sub.2 or Fv as well as single-domain and single-chain antibodies. A biologically active derivative of an antibody retains the ability to bind antigen.

The term “isolated DNA sequence” as described herein includes DNA whether single or double stranded. The sequence is isolated and/or purified (i.e. from its natural environment), in substantially pure or homogeneous form, free or substantially free of nucleic acid or genes of the species of interest or origin other than the promoter or promoter fragment sequence. The DNA sequence according to the present invention may be wholly or partially synthetic. The term “isolated” encompasses all these possibilities.

The term “low-pH conditions” as used herein refers to mildly acidic conditions with a pH range of 5.0 to 7.0, more preferably 5.8 to 6.6, and most preferably 6.2.

The term “oligonucleotide” as described herein refers to a probe or primer that is single-stranded DNA or RNA, or analogs thereof, that has a sequence of nucleotides that includes at least 14, preferably at least 20, more preferably at least 50, contiguous bases that are the same as (or the complement of) any 14 or more contiguous bases set forth in any of SEQ ID NO:1 to SEQ ID NO:4, [SEQ ID NO:9], [SEQ ID NO:11], [SEQ ID NO:13], [SEQ ID NO:15]. Preferred regions from which to construct probes include 5′ and/or 3′ coding regions of SEQ ID NO: 9, [SEQ ID NO:11], [SEQ ID NO:13], [SEQ ID NO:15]. In addition, the entire cDNA encoding region of SEQ ID NO:18 , or the entire sequence corresponding to SEQ ID NO: 20, may be used as a probe. Probes may be labeled by methods well-known in the art, as described hereinafter, and used in various diagnostic kits.

The term “operably linked” as described herein means joined as part of the same nucleic acid molecule, suitably positioned and oriented for transcription to be initiated from the same promoter.

The term “promoter′ as described herein refers to a sequence of nucleotides from which transcription may be initiated on DNA operably linked downstream (i.e. in the 3′ direction on the sense strand of double-stranded DNA).

The term “promoter activity” as described herein refers to the ability to initiate transcription.

The term “transcription cassette” as described herein refers to a nucleic acid sequence encoding a nucleic acid that is transcribed. To facilitate transcription, nucleic acid elements such as promoters and enhancers and transcriptional termination sequences are typically included in the transcription cassette.

We have found that SapM is an important determinant of mycobacterial pathogenesis.

In contrast to many bacterial acid phosphatases, the expression of SapM is not regulated by environmental inorganic phosphate concentration. Instead, it is regulated by pH. Pathogenic mycobacteria produce high levels of SapM under mildly acidic pH, and expression is maximal at pH 6.2. The sapM gene is present in pathogenic mycobacteria only. The sapM gene is selectively expressed in host macrophages, and is important for the virulence and pathogenesis of mycobacteria including M. tuberculosis. The expression of M. tuberculosis sapM is induced by the intracellular pH, and this gene is specific to pathogenic mycobacteria. SapM possess unique enzymatic activity and is an important determinant of mycobacterial pathogenesis. Unlike other bacterial acid phosphatases, SapM exhibits unusually high activity against GTP, which may affect the cellular GTP content and subsequently the balance of active to inactive forms of Rab proteins. This is particularly relevant since SapM is a small protein (28 kD) secreted extracellularly and is likely to gain access to the cytosol of the macrophages. The importance of SapM function in mycobacterial pathogenesis is further illustrated by the remarkable finding that SapM is present in pathogenic mycobacteria but not in nonpathogenic mycobacteria. SapM homologs are found in M. tuberculosis, M. bovis, M. bovis BCG, M. avium and M. marinum, all of which are pathogenic. In contrast, neither the SapM protein nor the sapM gene is detected in nonpathogenic, environmental mycobacteria such as M. smegmatis and M. chelonae.

Promoter Fragment

The present invention provides an isolated DNA sequence comprising a promoter or promoter fragment, the promoter or promoter fragment comprising a nucleotide sequence as provided in [SEQ ID NO:1], [SEQ ID NO:2], [SEQ ID NO:3], [SEQ ID NO:4]. The promoter or promoter fragment may comprise one or more fragments of the nucleotide sequence provided in [SEQ ID NO:1], [SEQ ID NO:2], [SEQ ID NO:3], [SEQ ID NO:4] sufficient to control expression of a nucleotide sequence of interest and which is inducible under low-pH conditions. The promoter or promoter fragment may comprise a sequence of nucleotides 5′ to position 498 as shown in [SEQ ID NO:1] in Mycobacterium tuberculosis or an equivalent sequence in other pathogenic mycobacterium or pathogenic fungi.

Restriction enzyme or nucleases may be used to digest the nucleic acid followed by an appropriate assay to determine the minimal nucleotide sequence required for promoter activity.

The promoter or promoter fragment may comprise one or more sequence motifs or elements conferring developmental and/or tissue-specific regulatory control of expression or regulatory control by an exogenous substrate or environmental condition. For example, the promoter or promoter-fragment may comprise a lung-specific regulatory control element or an element that is regulated by an exogenously added sugar, such as isopropyl-beta-D-thiogalactopyranoside.

The present invention extends to a promoter or promoter fragment which has a nucleotide sequence which is an allele, mutant, variant or derivative, by way of nucleotide addition, insertion, substitution or deletion. Alteration to the nucleotide sequence may be performed by techniques known to those skilled in the art. One or more alterations to a promoter or promoter fragment sequence or fragment of the present invention may increase or decrease promoter activity, or decrease the magnitude of the effect of a compound or protein cable of modulating the activity of the promoter or the promoter fragment.

Expression Vector

The present invention also provides an expression vector comprising a promoter or promoter fragment as disclosed herein. The promoter or promoter fragments can be cloned into a variety of vectors by means that are well known in the art. Such a vector may comprise a suitably positioned restriction site or other means for insertion of a nucleic acid sequence of interest into the vector that is operably linked to the promoter or promoter fragment of the current invention. Such a vector may comprise a suitably positioned restriction site or other means for insertion of a nucleotide sequence of interest into the vector that is operably linked to the promoter or promoter fragment.

Suitable vectors can be chosen or constructed containing appropriate regulatory sequences, including the promoter sequences or fragments thereof, terminator fragments, polyadenylation sequences, enhancer sequences, marker genes and other sequences as appropriate. For use in an assay or experiment, commercially available vectors such as the pET-series (Stratagene), pPRO-series (Clontech), pTet-series (Clontech), BacPAK systems (Clontech) may be employed. For use in gene therapy, vectors such as pT-Rex, pIND, pcDNA, pVAX1, and pEF may be employed. Expression vectors are useful to provide high levels of polypeptide expression. Cell cultures transformed with the DNA sequences of the current invention are useful as research tools particularly for studies of the effects of SapM on phagosome trafficking and maturation. Cell cultures may be used in overexpression and research according to numerous techniques known in the art. For example, a cell line (either an immortalized cell culture or a primary cell culture) may be transfected with a vector containing a transcription cassette of the current invention which permits the assessment of the levels of nucleic acid of interest produced, the polypeptide, the functionality and the phenotype of the cells produced.

For further details see, for example, Molecular Cloning: a Laboratory Manual: 2^{nd} edition, Sambrook et al., 1989 Cold Spring Harbor Laboratory Press. Procedures for introducing DNA into cells depends on the host used, but are well known, such as electroporation and calcium phosphate transformation methods.

Host Cell

A further aspect of the present invention provides a host cell containing a transcription cassette of the current invention. Particularly desirable host cells include M. tuberculosis, M. marinum, M. bovis, M. bovis BCG, M. smegmatis, members of the M. avium complex, E. coli (and derivatives such as E. coli BL21(DE3) ), insect cell lines, such as Sf21, for protein overexpression, and mammalian cell lines, such as RAW, and J774. Examples of suitable genes to be expressed under control of the sapM promoter are: antigen 85 genes (fbpA, fbpB, fbpC), 19-Kd lipoprotein gene (Ippo) and alpha-crystallin gene (acr).

Methods known in the art for transformation, include, but are not limited to, electroporation, rubidium chloride, calcium chloride, calcium phosphate or chloroquine transfection, viral infection, phage transduction and microinjection, and the use of cationic lipid and lipid/amino acid complexes, or of liposomes, or a large variety of other commercially available, and readily synthesized transfection adjuvants, are useful to transfer a SapM nucleic acid molecule into host cells.

Host cells are cultured in conventional nutrient media. The media may be modified as appropriate for inducing promoters, amplifying nucleic acid sequences of interest or selecting transformants. The culture conditions, such as temperature, composition and pH will be apparent. After transformation, transformants may be identified on the basis of selectable phenotype.

Method of Diagnosing

Detection of sapM nucleic acid, SapM protein, or SapM phosphatase activity is useful as a screening tool for the presence of a pathogenic mycobacterial or a pathogenic fungal infection or to monitor the progression of a pathogenic mycobacterial or a pathogenic fungal infection.

The present invention relates to a method for the diagnosis of a pathogenic mycobacterial infection or a pathogenic fungal infection in a subject comprising:
a) obtaining a biological sample from a subject; and
b) analyzing the sample for the presence of antibodies specific to SapM [SEQ ID NO:10], [SEQ ID NO:12;],[SEQ ID NO:14], [SEQ ID NO:16] wherein detection of antibodies specific to SapM [SEQ ID NO:10], [SEQ ID NO:12], [SEQ ID NO:14], [SEQ ID NO:16] is indicative of the presence of the pathogenic mycobacterial or pathogenic fungal infection.

The sample may be analyzed for the presence of antibodies specific to SapM by using SapM in immunoassays wherein SapM can be utilized in liquid phase or bound to a solid phase carrier. In addition, SapM can be detectably labeled in various ways for use in immunoassays with anti-SapM antibodies. The preferred immunoassays for detecting anti-SapM antibodies using the methods of this invention include radioimmuno-assays, enzyme-linked immunosorbent assays (ELISA), or other assays known in the art, such as immunofluorescent assays, chemiluminescent assays, or bioluminescent assays.

The present invention relates to a method for the diagnosis of a pathogenic mycobacterial infection or a pathogenic fungal infection in a subject comprising:
a) obtaining a nucleic acid sample from a subject; and
b) analyzing the sample for the presence of nucleic acid encoding SapM, wherein detection of sapM is indicative of the presence of the pathogenic mycobacterial or pathogenic fungal infection.

The sample may be analyzed for the presence of nucleic acid encoding SapM by, for example, PCR analysis, DNA sequencing, SSCP analysis, or RFLP analysis.

In another embodiment the present invention relates to a method for the diagnosis of a pathogenic mycobacterial infection or a pathogenic fungal infection in a subject comprising:
a) obtaining a biological fluid sample from a subject; and
b) analyzing the sample for the presence of SapM phosphatase activity, wherein detection of SapM phosphatase activity is indicative of the presence of the pathogenic mycobacterial or pathogenic fungal infection.

The sample may be analyzed for the presence of SapM phosphatase activity by performing the acid phosphatase assay using GTP and/or NADPH as cofactors and p-nitrophenylphosphate as substrate.

Measurement of sapM nucleic acid, SapM protein, or SapM phosphatase activity may be used not only for the diagnosis of a pathogenic mycobacterial or a pathogenic fungal infection but also to monitor therapeutic response, assess prognosis, assess patient disease risk and to monitor the success of disease preventative interventions in patients at risk.

Other assays (as well as variations of the above assay) will be apparent from the description of this invention and techniques.

Method of Screening

The present invention also relates to a method of screening a synthetic compound or protein that regulates or modulates promoter activity of the promoter or promoter fragment DNA of the present invention. A method of identifying a synthetic compound or protein that regulates or modulates the promoter activity of the SapM promoter or promoter fragment comprises:
a) employing or providing a nucleic acid construct comprising a functional part of the SapM promoter [SEQ ID NO:1], [SEQ ID NO:2], [SEQ ID NO:3], [SEQ ID NO:4] the functional part of the promoter being operably linked to a reporter gene capable of producing a detectable signal;
b) exposing the nucleic acid construct to candidate compounds or proteins; and
c) comparing the signal produced in the absence of the compound or protein tested.

The reporter gene preferably encodes an enzyme which catalyses a reaction that produces a detectable signal. Many examples are known, for example, gfp encoding the green fluorescent protein (and gfp variants encoding enhanced green, blue/cyan and yellow fluorescent proteins), DsRed encoding the red fluorescent protein, lacZ encoding Beta-galactosidase, gus encoding Beta-glucomnidase, luxAB (or lucCDABE) encoding bacterial luciferase, luc encoding firefly luciferase, and phoA encoding alkaline phosphatase. Those skilled in the art are well aware of a multitude of possible reporter genes and assay techniques that may be used to determine gene activity. Any suitable reporter/assay may be used and it should be appreciated that no particular choice is essential to or a limitation of the present invention. The reporter gene may be used in an in vitro or an in vivo expression system. In addition to a promoter or a promoter fragment, expression generally requires the presence of a translational initiation region and transcriptional and translational termination regions.

Another embodiment of the current invention relates to a method of screening a synthetic compound or protein that regulates or modulates SapM phosphatase activity comprising:
a) incubating a mixture comprising SapM [SEQ ID NO:10], [SEQ ID NO:12], [SEQ ID NO:14], [SEQ ID NO:16], a SapM substrate, and the compound to be tested;
b) measuring the phosphatase activity; and
c) comparing the phosphatase activity in the absence of the compound or protein to be tested.

The substrate may comprise pNPP, GTP or NADPH.

Another embodiment of the current invention relates to a method of screening a synthetic compound or protein that regulates or modulates SapM secretion comprising:
a) exposing mycobacterium cells to a compound or protein to be tested, wherein the mycobacterium cells secrete SapM [SEQ ID NO:10]; [SEQ ID NO:12], [SEQ ID NO:14], [SEQ ID NO:16];
b) detecting the presence or activity of SapM [SEQ ID NO:10], [SEQ ID NO:12], [SEQ ID NO:14], [SEQ ID NO:16] secreted by the mycobacterium cells; and
c) comparing the secretion of SapM [SEQ ID NO:10], [SEQ ID NO:12], [SEQ ID NO:14], [SEQ ID NO:16] in the absence of the compound or protein to be tested.

“Modulation”, “modulates” and “modulating” can refer to an increase in expression or activity, a decrease in expression or activity, a change in the type or kind of expression or activity present, a complete cessation of expression or activity (i.e., an absence of expression or activity), or the instigation of expression or activity. Suitable compounds that may be used include but are not limited to proteins, nucleic acids, small molecules, hormones, antibodies, peptides, antigens, cytokines, growth factors, pharmacological agents including chemotherapeutics, carcinogenics, or other cells (i.e. cell-cell contacts). Cells may also be screened for the effects of environmental or physiological factors such as radiation, action potentials, etc. on normal gene expression.

Other assays (as well as variations of the above assay) will be apparent from the description of this invention and techniques such as those disclosed in U.S. Pat. Nos. 5,851,788, 5,736,337 and 5,767,075 which are incorporated by reference in their entirety. For example, the test compound levels may be either fixed or increased, and a plurality of compounds or proteins may be tested at a single time.

Kits

The invention includes a kit for detecting the presence of SapM nucleic acid molecule, comprising at least one probe of the invention. Kits may be prepared according to known techniques, for example, see U.S. Pat. Nos. 5,851,788 and 5,750,653. The kit preferably includes reagents suitable for the hybridization of the probe to a complementary nucleic acid sequence. The invention also includes a kit for detecting the presence of SapM protein, comprising at least one anti-SapM antibody of the invention. Kits may be prepared according to known techniques, for example, see U.S. Pat. Nos. 5,851,788 and 5,750,653.

The kit preferably includes an antibody, a medium suitable for the formation of an immunological complex between the antibody and a polypeptide recognized by the antibody and a reagent capable of detecting the immunological complex to ascertain the presence of SapM or a similar polypeptide in a biological sample. Further background on the use of antibodies is provided, for example in U.S. Pat. Nos. 5,695,931 and 5,837,472 which are incorporated by reference in their entirety.

Preparation of Antibodies

The invention includes an isolated antibody immunoreactive with a polypeptide of the invention. Antibodies are preferably generated against epitopes of native SapM [SEQ ID NO:10], [SEQ ID NO:12]; [SEQ ID NO:14], [SEQ ID NO:16] or synthetic peptides of SapM [SEQ ID NO:10], [SEQ ID NO:12], [SEQ ID NO:14], [SEQ ID NO:16]. The antibody may be labeled with a detectable marker or unlabeled. The antibody is preferably a monoclonal antibody or a polyclonal antibody. SapM antibodies can be employed to screen organisms containing SapM polypeptides. The antibodies are also valuable for immuno-purification of polypeptides from crude extracts. For example, one may contact a biological sample with the antibody under conditions allowing the formation of an immunological complex between the antibody and a polypeptide recognized by the antibody and detecting the presence or absence of the immunological complex whereby the presence of SapM or a similar polypeptide is detected in the sample. The invention also includes compositions, preferably including the antibody, a medium suitable for the formation of an immunological complex between the antibody and a polypeptide recognized by the antibody and a reagent capable of detecting the immunolgical complex to ascertain the presence of SapM or a similar polypeptide.

To recognize SapM, one may generate antibodies against a range of unique epitopes throughout the molecule, for example [SEQ ID NO:22] (NDMHDGSI). One could generate antibodies that target the N-terminal signal sequence, to block the secretion of SapM. In addition, these antibodies, or other antibodies directed against other SapM epitopes, could block SapM activity by leading to enhanced SapM clearance/degradation, with a concomitant decrease in SapM phosphatase activity.

Monoclonal and polyclonal antibodies are prepared according to the description in this application and techniques known in the art. For examples of methods of the preparation and uses of monoclonal antibodies, see U.S. Pat. Nos. 5,688,681, 5,688,657, 5,683,693, 5,667,781, 5,665,356, 5,591,628, 5,510,241, 5,503,987, 5,501,988, 5,500,345 and 5,496,705 that are incorporated by reference in their entirety. Examples of the preparation and uses of polyclonal antibodies are disclosed in U.S. Pat. Nos. 5,512,282, 4,828,985, 5,225,331 and 5,124,147 which are incorporated by reference in their entirety.

The invention also includes methods of using the antibodies. For example, the invention includes a method for detecting the presence of a SapM polypeptide such as [SEQ ID NO:10], [SEQ ID NO:12], [SEQ ID NO:14], [SEQ ID NO:16] or [SEQ ID NO:22] by: a) contacting a sample containing one or more polypeptides with an antibody of the invention under conditions suitable for the binding of the antibody to polypeptides with which it is specifically reactive; b) separating unbound polypeptides from the antibody; and c) detecting antibody which remains bound to one or more of the polypeptides in the sample.

Immunogenic Compositions

One skilled in the art will appreciate that suitable methods of administering the immunogenic compositions of the present invention are available. Preferably, the compositions are administered parenterally, e.g., intravenously, intraarterially, intrathecally, subcutaneously, intradermally or intramuscularly. The requirements for effective pharmaceutical carriers for parenteral compositions are well known to those of ordinary skill in the art (see, e.g. Banker and Chalmers (eds.), Pharmaceutics and Pharmacy Practice, J.B. Lippincott Company, Philadelphia, Pa., (1982) and Toissel, ASHIP Handbook on Injectable Drugs (4^{th} ed.)). Such solutions can contain anti-oxidants, buffers, bacteriostats, and solutes that render the formulations isotonic with the blood of the intended recipient, and aqueous and non-aqueous sterile suspensions that can include suspending agents, solubilizers, thickening agents, stabilizers, and preservatives. Compounds may be administered in a physiologically acceptable diluent in pharmaceutical carrier, such as sterile liquid or mixture of liquids, including water, saline, aqueous dextrose and related sugar solutions, an alcohol, such as ethanol, isopropanol, or hexadecyl alchohol, glycols such as propylene glycol or polyethylene glycol, dimethylsulfoxide, glycerol ketals such as 2,2-dimethyl-1,3-dioxolane4-methanol, ethers, such as poly(ethyleneglycol) 400, an oil, a fatty acid, a fatty acid ester or glyceride or an acetylated fatty acid glyceride with or without the addition of a pharmaceutically acceptable surfactant, such as soap or a detergent, suspending agent, such as pectin, carbomers, methyl cellulose, hydroxypropylmethylcellulose or caboxymethylcellulose or emulsifying agents and other pharmaceutical adjuvants.

Oils useful in parenteral formulations include petroleum, animal, vegetable, or synthetic oils. Specific examples of oils useful in such formulations include peanut, soybean, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents including, cationic detergents, anionic detergents, nonionic detergents, amphoteric detergents and mixtures thereof.

The parenteral formulations typically will contain from about 0.5 to about 25% by weight of the active ingredient in solutions. Preservatives and buffers may be used. Parenteral formulations can be presented in unit-dose or multi-dose sealed containers and can be lyophilized. Extemporaneous injection solutions and suspensions can be prepared from sterile powders, granules, and tablets. Topical formulations, including those that are useful for transdermal drug release, are well-known to those of skill in the art and are suitable in the context of the present invention for application to the skin.

Formulations suitable for oral administration can consist of liquid solutions, capsules, powders, suspensions and suitable emulsions. Liquid formulations may include diluents, such as water and alcohol. Capsule forms can be of the ordinary hard- or soft-shelled gelatin type containing, for example, surfactants, lubricants, and inert fillers such as lactose, sucrose, calcium phosphate, and cornstarch. Tablet forms can include excipients, colorants, diluents, buffering agents, disintegrating agents, moistening agents, preservatives, flavoring agents and pharmacologically compatible excipients.

The immunogenic compositions of the current invention can be made into aerosol formulations to be administered by inhalation. Formulations suitable for aerosol administration may include surfactants and propellant. A carrier may also be included for intranasal delivery.

The compositions of the present invention may also be administered as suppositories by mixing with a variety of bases, such as emulsifying bases or water-soluble bases. Formulations suitable for vaginal administration may be presented as pessaries, tampons, creams, gels, pastes, foams or spray formulas.

The concentration of SapM protein or polypeptide fragment thereof for immunogenic compositions can vary widely, i.e., from less than about 1% usually at or at least about 10% to as much as 20 to 50% or more by weight and will be selected primarily be fluid volumes, viscosities, etc., in accordance with the particular mode of administration selected. Actual methods for preparing parenterally administrable compositions will be know or apparent to those skilled in the art and are described in more detail, for example, Remington's Pharmaceutical Science (17^{th} ed., Mack Publishing company, Easton, Pa., (1985).

The compounds of the present invention may be formulated as inclusion complexes or liposomes. Liposomes serve to target the SapM protein or polypeptide fragment thereof peptides to a particular tissue. Liposomes can also increase the half-life of the compositions.

Vaccine Preparation and Use

Immunogenic compositions, suitable to be used as vaccines, may be prepared from SapM protein and polypeptide fragments thereof and nucleic acid molecules encoding SapM proteins and polypeptide fragments thereof. For examples of methods of the preparation and uses of vaccines, see U.S. Pat. Nos. 4,601,903,4,599,231, 4,599,230, and 4,596,792 that are incorporated by reference in their entirety.

Immunogenic compositions including vaccines may be prepared as injectables, either as liquid solutions or suspensions; solid forms suitable for solution in, or suspension in, liquid prior to injection may also be prepared. The preparation may also be emulsified, or the protein encapsulated in liposomes. The live immunogenic ingredients are often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, and/or adjuvants that enhance the effectiveness of the vaccine. Examples of adjuvants which may be effective include but are not limited to: aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80™ emulsion. The effectiveness of an adjuvant may be determined by measuring the amount of antibodies directed against an immunogenic polypeptide containing a SapM antigenic sequence resulting from administration of the SapM vaccines that are also comprised of the various adjuvants.

The vaccines are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1%-2%. Oral formulations include such normally employed excipients as, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10%-95% of active ingredient, preferably 25%-70%. The vaccines are administered in a manner compatible with the dosage formulation, and in such amount as will be prophylactically and/or therapeutically effective.

The vaccine may be given in a single dose schedule, or preferably in a multiple dose schedule. A multiple dose schedule is one in which a primary course of vaccination may be with 1-10 separate doses, followed by other doses given at subsequent time intervals required to maintain and or reinforce the immune response, for example, at 14 months for a second dose, and if needed, a subsequent dose(s) after several months. The dosage regimen will also, at least in part, be determined by the need of the individual and be dependent upon the judgment of the practitioner.

In addition, the vaccines may be administered in conjunction with other immunoregulatory agents, for example, immune globulins.

Nucleic Acid Molecules

Functionally Equivalent Nucleic Acid Molecule or Polypeptide Sequence

The term “isolated DNA sequence” refers to a DNA sequence the structure of which is not identical to that of any naturally occurring DNA sequence or to that of any fragment of a naturally occurring DNA sequence spanning more than three separate genes. The term therefore covers, for example, (a) DNA which has the sequence of part of a naturally occurring genomic DNA molecule; (b) a DNA sequence incorporated into a vector or into the genomic DNA of a prokaryote or eukaryote, respectively, in a manner such that the resulting molecule is not identical to any naturally occurring vector or genomic DNA; (c) a separate molecule such as cDNA, a genomic fragment, a fragment produced by reverse transcription of polyA RNA which can be amplified by PCR, or a restriction fragment; and (c) a recombinant DNA sequence that is part of a hybrid gene, i.e., a gene encoding a fusion protein. Specifically excluded from this definition are nucleic acids present in mixtures of (i) DNA molecules, (ii) transfected cells, and (iii) cell clones, for example, as these occur in a DNA library such as a cDNA or genomic DNA library.

Modifications in the DNA sequence, which result in production of a chemically equivalent or chemically similar amino acid sequence, are included within the scope of the invention. Modifications include substitution, insertion or deletion of nucleotides or altering the relative positions or order of nucleotides. Variants of the polypeptides of the invention may occur naturally by mutation for example, or may be made with polypeptide engineering techniques such as site directed mutagenesis for example, which are well known in the art for substitution of amino acids. For example, a hydrophobic residue such as alanine may be substituted with a more hydrophobic residue such as leucine, valine or isoleucine. A negatively charged amino acid such as aspartic acid may be substituted for glutamic acid. A positively charged amino acid such as lysine may be substituted for another positively charged amino acid such as arginine.

Therefore, the invention includes polypeptides having conservative changes or substitutions in amino acid sequences. Conservative substitutions insert one or more amino acids, which have similar chemical properties as the replaced amino acids. The invention includes sequences where conservative substitutions are made that do not destroy SapM activity.

The invention also includes polypeptide fragments of the polypeptides of the invention that may be used to confer SapM activity if the fragments retain activity. The invention also includes polypeptide fragments of the polypeptides of the invention which may be used as a research tool to characterize the polypeptide or its activity. Such polypeptides preferably consist of at least 5 amino acids. In preferred embodiments, they may consist of 6 to 10, 11 to 15, 16 to 25, 26 to 50, 51 to 75, 76 to 100 or 101 to 250 amino acids of the polypeptides of the invention (or longer amino acid sequences). The fragments preferably have SapM activity. Fragments may include sequences with one or more amino acids removed, for example, C-terminus amino acids in a SapM sequence.

This invention also includes sequences where conservative substitutions are made that increase or decrease the activity of SapM.

Polypeptides comprising one or more d-amino acids are contemplated within the invention. Also contemplated are polypeptides where one or more amino acids are acetylated at the N-terminus. Those of skill in the art recognize that a variety of techniques are available for constructing polypeptide mimetics with the same or similar desired SapM activity as the corresponding polypeptide compound of the invention but with more favourable activity than the polypeptide with respect to solubility, stability, and/or susceptibility to hydrolysis and proteolysis. See, for example, Morgan and Gainor, (1989) Ann. Rep. Med. Chem., 24:243-252. Examples of polypeptide mimetics are described in U.S. Pat. No. 5,643,873. Other patents describing how to make and use mimetics include, U.S. Pat. Nos. 5,786,322, 5,767,075, 5,763,571, 5,753,226, 5,683,983, 5,677,280, 5,672,584, 5,668,110, 5,654,276, 5,643,873. Mimetics of the polypeptides of the invention may also be made according to other techniques known in the art, for example, by treating a polypeptide of the invention with an agent that chemically alters a side group by converting a hydrogen group to another group such as a hydroxy or amino group. Mimetics preferably include sequences that are either entirely made of amino acids or sequences that are hybrids including amino acids and modified amino acids or other organic molecules.

The invention also includes hybrid nucleic acid molecules and polypeptides, for example where a sapM DNA sequence from one species is combined with a nucleotide sequence from a sequence of plant, mammal, bacteria or yeast to encode a fusion polypeptide. The invention includes a fusion protein having at least two components, wherein a first component of the fusion protein comprises a polypeptide of the invention, preferably a full length SapM polypeptide (or a portion thereof, see below). The second component of the fusion protein preferably comprises a tag, for example GST, an epitope tag or an enzyme. The fusion protein may also comprise a histochemical or cytochemical marker such as lacZ, alkaline phosphatase, or horseradish peroxidase, or a fluorescent marker such as GFP or one of its derivatives. The invention also includes a composition comprising all or part of an isolated DNA molecule (preferably sapM [SEQ ID NO:9], [SEQ ID NO:11], [SEQ ID NO:13], [SEQ ID NO:15]) of the invention with or without a carrier, preferably in a composition for cell transformation. The invention also includes a composition comprising an isolated SapM polypeptide (preferably SapM [SEQ ID NO:10], [SEQ ID NO:12], [SEQ ID NO:14], [SEQ ID NO:16] or [SEQ ID NO: 22]) with or without a carrier, preferably for studying or modulating polypeptide activity.

Sequence Identity

The invention includes modified nucleic acid molecules with a sequence identity at least about: >17%, >20%, >30%, >40%, >50%, >60%, >70%, >80% or >90% more preferably at least about >95%, >99.5%, to the DNA sequences provided in [SEQ ID NO: 1] to [SEQ ID NO: 4] or [SEQ ID NO:9], [SEQ ID NO:11], [SEQ ID NO:13], [SEQ ID NO:15] (ora partial sequence thereof or their complementary sequence). Preferably about 1, 2, 3,4, 5, 6 to 10, 10 to 25, 26 to 50, 51 to 100, or 101 to 250 nucleotides are modified. Sequence identity is most preferably assessed by the algorithm of the BLAST version 2.1 program advanced search (parameters as above). BLAST is a series of programs that are available online at http//www.ncbi.nlm.nih.gov/BLAST.

References to BLAST searches are:

References to BLAST searches are:

Altschul, S. F., Gish, W., Miller, W., Myers, E. W. & Lipman, D. J. (1990) “Basic local alignment search tool.” J. Mol. Biol. 215:403-410.

Gish, W. & States, D. J. (1993) “Identification of protein coding regions by database similarity search.” Nature Genet. 3:266-272.

Madden, T. L., Tatusov, R. L. & Zhang, J. (1996) “Applications of network BLAST server” Meth. Enzymol. 266:131-141.

Altschul, S. F., Madden, T. L., Schaffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402.

Zhang, J. & Madden, T. L. (1997) “PowerBLAST: A new network BLAST application for interactive or automated sequence analysis and annotation.” Genome Res. 7:649-656.

The polypeptides encoded by the homologous sapM nucleic acid molecule in other species will have amino acid sequence identity at least about: >20%, >25%, >28%, >30%, >40% or >50% to an amino acid sequence as provided in [SEQ ID NO:10], [SEQ ID NO:12], [SEQ ID NO:14], [SEQ ID NO:16] (or a partial sequence thereof). Some species may have polypeptides with a sequence identity of at least about: >60%, >70%, >80% or >90%, more preferably at least about: >95%, >99% or >99.5% to all or part of an amino acid sequence in as shown in [SEQ ID NO:10], [SEQ ID NO:12], [SEQ ID NO:14], [SEQ ID NO:16] (or a partial sequence thereof). Identity is calculated according to methods known in the art. Sequence identity is most preferably assessed by the BLAST version 2.1 program advanced search (parameters as above). Preferably about: 1, 2, 3,4, 5, 6 to 10, 10 to 25, 26 to 50, 51 to 100, or 101 to 250 nucleotides or amino acids are modified.

The invention includes nucleic acid molecules with mutations that cause an amino acid change in a portion of the polypeptide not involved in providing SapM activity or an amino acid change in a portion of the polypeptide involved in providing SapM activity so that the mutation increases or decreases the activity of the polypeptide.

The sequences of the invention can be prepared according to numerous techniques. The invention is not limited to any particular preparation means. For example, the nucleic acid molecules of the invention can be produced by cDNA cloning, genomic cloning, cDNA synthesis, polymerase chain reaction (PCR) or a combination of these approaches (Current Protocols in Molecular Biology, F. M. Ausbel et al., 1989). Sequences may be synthesized using well-known methods and equipment, such as automated synthesizers.

Hybridization

Other functional equivalent forms of the SapM DNA molecules can be isolated using conventional DNA-DNA or DNA-RNA hybridization techniques. These nucleic acid molecules and the SapM sequences can be modified without significantly affecting their activity.

The present invention also includes nucleic acid molecules that hybridize to one or more of the DNA sequences provided in [SEQ ID NO:1] to [SEQ ID NO:4] and [SEQ ID NO:9], [SEQ ID NO:11], [SEQ ID NO:13], [SEQ ID NO:15] (or a partial sequence thereof or their complementary sequence) and that encode peptides or polypeptides exhibiting substantially equivalent activity as that of a SapM polypeptide produced by the DNA in [SEQ ID NO:10], [SEQ ID NO:12], [SEQ ID NO:14], [SEQ ID NO:16] Such nucleic acid molecules preferably hybridize to all or a portion of sapM or its complement under low, moderate (intermediate), or high stringency conditions as defined herein (see Sambrook et al. (most recent edition) Molecular Cloning: A Laboratory Manual, Cold Spring Harbor Laboratory Press, Cold Spring Harbor, N.Y.; Ausubel et al. (eds.), 1995, Current Protocols in Molecular Biology, (John Wiley & Sons, NY)). The portion of the hybridizing nucleic acids is typically at least 15 (e.g. 20, 25, 30 or 50) nucleotides in length. The hybridizing portion of the hybridizing nucleic acid is at least 80% e.g. at least 95% or at least 98% identical to the sequence or a portion or all of a nucleic acid encoding a SapM polypeptide, or its complement. Hybridizing nucleic acids of the type described herein can be used, for example, as a cloning probe, a primer (e.g. a PCR primer) or a diagnostic probe. Hybridization of the oligonucleotide probe to a nucleic acid sample typically is performed under stringent conditions. Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g. SSC or SSPE). Then, assuming that 1% mismatching results in a 1 degree Celsius decrease in the Tm, the temperature of the final wash in the hybridization reaction is reduced accordingly (for example, if sequences having greater than 95% identity with the probe are sought, the final wash temperature is decreased by 5 degrees Celsius). In practice, the change in Tm can be between 0.5 degrees Celsius and 1.5 degrees Celsius per 1% mismatch. Low stringency conditions involve hybridizing at about: 1×SSC, 0.1% SDS at 50° C. High stringency conditions are: 0.1×SSC, 0.1% SDS at 65° C. Moderate stringency is about 1×SSC, 0.1% SDS at 60 degrees Celsius. The parameters of salt concentration and temperature can be varied to achieve the optimal level of identity between the probe and the target nucleic acid. The present invention also includes nucleic acid molecules from any source, whether modified or not, that hybridize to genomic DNA, cDNA, or synthetic DNA molecules that encode the amino acid sequence of a SapM polypeptide, or genetically degenerate forms, under salt and temperature conditions equivalent to those described in this application, and that code for a peptide, or polypeptide that has SapM activity. Preferably the polypeptide has the same or similar activity as that of a SapM polypeptide. A nucleic acid molecule described above is considered to be functionally equivalent to a sapM nucleic acid molecule of the present invention if the polypeptide encoded by the nucleic acid molecule is recognized in a specific manner by a SapM-specific antibody, including, but not restricted to, the antibodies listed in this application.

Expression of sapM is Induced in M. bovis BCG Grown on Ammonium Chloride

Bacterial acid and alkaline phosphatases are typically regulated by environmental inorganic phosphate concentration (reviewed in (Rossolini et al., 1998)). The expression of these enzymes, which catalyze the hydrolysis of exogenous sources of phosphorylated components into inorganic phosphate, is induced under phosphate starvation. This essential limiting nutrient is then transported into the cell by membrane permeases. A recent study showed that SapM of M. tuberculosis is an acid phosphatase secreted to the culture medium (Saleh and Belisle, 2000). To determine if SapM is involved in phosphate assimilation, the effect of environmental phosphate concentration on SapM expression and activity were examined. Four M. bovis BCG strains, organisms closely related to M. tuberculosis, were grown in Sauton medium containing three different concentrations of inorganic phosphate, 0.05, 0.5, and 5.0 g/L, and the acid phosphatase activity in the culture filtrate protein (CFP) fractions was assayed. Under these conditions, acid phosphatase activity is low, ranging from 40- to 130-nmol·hr⁻¹·mg⁻¹ (total protein). In contrast to most bacterial acid phosphatases, the acid phosphatase activity in the CFP fraction of BCG cultures was not regulated by the phosphate concentration, and phosphate starvation did not increase its activity (FIG. 1A). Starvation of carbon source in the culture medium also did not affect the enzyme activity (FIG. 1A).

Interestingly, high levels of acid phosphatase activity were detected in BCG grown in media containing ammonium chloride (NH₄Cl) as the nitrogen source (FIG. 1A). The original Sauton medium contains 27 mM of asparagine as the primary nitrogen source. Asparagine was substituted by NH₄Cl at the equal molar concentration and the fresh medium was adjusted to pH 7.4. All BCG strains tested, BCG-Birkhaug, -Japan, -Frappier and -Pasteur, grow normally in the ammonium-containing Sauton medium. The CFP fractions prepared under these conditions exhibited much higher levels of acid phosphatase activity than those grown in the original Sauton (FIG. 1A). BCG-Birkhaug exhibited a dramatic 25-fold increase, from 136- to 3,459-nmol·hr⁻¹·mg⁻¹. Other BCG strains exhibited 5- to 10-fold increases of acid phosphatase activity. Adjustment of phosphate or glycerol concentrations in the ammonium-containing Sauton media did not change the enzyme activity level (data not shown).

It was previously shown that SapM is the only acid phosphatase detected in the extracellular fraction of M. tuberculosis cultures (Saleh and Belisle, 2000). To determine if SapM contributes to the observed increase of acid phosphatase activity, CFP fractions were analyzed by SDS-PAGE. As shown in FIG. 1B, a protein band, visualized by silver staining, was produced by BCG-Birkhaug grown in ammonium-containing Sauton, and was not detected in culture grown in normal Sauton medium. The mobility of this protein corresponds to the molecular weight of the mature SapM protein (˜28 kD) and to that of partially purified SapM. To confirm the identity of this protein, an antibody raised against a synthetic peptide corresponding to amino acid residues 201-208 of SapM protein was made and Western blotting performed. Indeed, the 28-kD protein reacted with the anti-SapM antisera, indicating that this protein is SapM (FIG. 1B). For other BCG strains, SapM protein is less visible, which correlates with the lower level of enzyme activity detected in these strains (FIG. 1A).

SapM of M. bovis BCG is Selectively Expressed at Mildly Acidic pH

It was not immediately clear why SapM expression would be induced in media containing NH₄Cl. Although our initial hypothesis was that sapM was regulated by the availability of nitrogen source, nitrogen starvation did not alter the SapM expression or activity (data not shown). However, uptake of ammonia (NH₄⁺) is accomplished by a membrane transporter, which may couple ammonia uptake to H⁺ export (Westhoff et al., 2002). This would result in a gradual acidification of the culture medium. Indeed, measuring the pH of the spent BCG culture media revealed that cell growth leads to acidification of Sauton medium containing NH₄Cl (i.e., the pH changed from 7.4 to 5.8-6.2), whereas the pH of asparagine-containing Sauton medium remains at 7.4-7.8.

We therefore hypothesized that the acidification of the culture medium is responsible for the induction of SapM expression. To test this, BCG-Birkhaug was grown in asparagine-containing Sauton medium (pH 7.4), then portions of cells were washed and cultured in the same medium except the pH was adjusted to 7.0, 6.6, 6.2, and 5.8 with appropriate buffers. Acid phosphatase activity from the CFP fractions of these cultures was determined and revealed a correlation between the level of acid phosphatase activity and the pH of the culture media (FIG. 2A). At pH 7.0-7.4, the level of acid phosphatase activity is very low. Under mildly acidic conditions (i.e., pH 5.8-6.6), a high level of acid phosphatase activity was detected and was maximal at pH 6.2 (FIG. 2A). SDS-PAGE and Western blotting analyses-confirmed that SapM expression is induced at pH 5.8-6.6 (FIG. 2B), which correlates well with the high levels of enzyme activity at the same pH. These results indicate that sapM is selectively expressed at mildly acidic pH. The level of SapM activity detected in BCG-Birkhaug grown at pH 5.8-6.2 is similar to that grown in ammonium-containing Sauton medium (compare FIGS. 1A & 2A), suggesting that the induction of SapM expression by ammonium described above actually reflects the effect of pH.

At pH 7.0-7.4, SapM was not detected either by silver staining or Western blotting (FIG. 2B), suggesting that SapM level is very low or below detection limits, which is consistent with the residual acid phosphatase activity detected at these pH values (FIG. 2A). Alternatively, the residual acid phosphatase activity may originate from a small fraction of cell surface-associated acid phosphatase that leaks to the external medium during cell growth. Other than SapM, only one gene product of M. tuberculosis, Rv2577, is predicted to be an acid phosphatase (Cole et al., 1998). However, Rv2577 does not posses an export signal sequence and appears to be cell surface associated (Braibant and Content, 2001). The activities of cell surface-associated acid phosphatase and alkaline phosphatase of BCG were not affected by the change of pH in the culture medium (FIG. 2C), suggesting that the pH-dependent expression is a specific property of SapM.

Cloning and Expression of M. tuberculosis sapM in Mycobacterium smegmatis

SapM was originally identified by biochemical approaches from the extracellular medium of M. tuberculosis H37Rv grown in glycerol-alanine-salts (GAS) medium (Saleh and Belisle, 2000). Like our ammonium-containing Sauton medium, GAS contains NH₄Cl as a nitrogen source and the spent GAS medium is mildly acidic (data not shown). To confirm that the SapM of M. tuberculosis shares the same property as that of M. bovis BCG, a DNA fragment containing the sapM gene (Rv3310) was cloned into shuttle vector pMD31 to generate pSAP (FIG. 3A), which was then transformed into Mycobacterium smegmatis mc²-155. The sapM gene is absent from the M. smegmatis chromosome and SapM activity was not detected in the extracellular CFP fraction (see below). However, when recombinant M. smegmatis containing sapM was grown in Sauton medium containing NH₄Cl, SapM protein was detected upon SDS-PAGE and Western blotting analyses of the CFP fraction (FIG. 3B). SapM was absent from the pSAP strain grown in Sauton containing asparagine, and from the control strain containing the cloning vector pMD31. These results indicated that M. tuberculosis sapM gene can be expressed and secreted from M. smegmatis.

To examine if M. tuberculosis sapM is regulated by pH, mc²-155/pSAP was grown in Sauton medium (containing asparagine) adjusted to various pH levels, as described above. The enzyme activity and the protein concentration of SapM correlated with each other, and both were dependent on the pH of the culture medium (FIGS. 4A-C). High levels of SapM expression and activity were detected at pH 5.8-6.6, which is in agreement with results obtained in M. bovis BCG. The control strain containing the cloning vector alone showed residual acid phosphatase activity, which is likely due to leakage of the cell surface-associated acid phosphatase, and is not pH inducible.

The M. tuberculosis sapM Gene is Selectively Expressed Inside Macrophages

Our in vitro studies demonstrate that sapM is selectively expressed at pH 5.8-6.6, a pH level that coincides with mildly acidic mycobacteria-containing phagosome (pH 6.1-6.5) (Sturgill et al., 1994), which suggests that sapM expression is induced within macrophages. To demonstrate that sapM is expressed in vivo, two transcriptional fusions were constructed. pSAPM-GFP contains 560 bp upstream of the sapM coding region and the sapM gene truncated 67 bp from its 3′ end. pSAPC-GFP contains the same 560 bp sapM promoter fragment, the entire sapM gene and the sapC gene (Rv3311) truncated 183 bp from its 3′ end (FIG. 5A). The sapM and sapC genes appear to be co-transcribed. In both vectors, the truncated genes are transcriptionally fused to a promoterless mutant green fluorescent protein (mGFP) (Barker et al., 1998). These constructs, together with the cloning vector pFPV27, were transformed into Mycobacterium marinum, a fish pathogen that also produces SapM from a chromosomal loci (see below). Recombinant M. marinum strains grown in asparagine-containing Sauton medium (pH 7.4), in which the sapM expression is not induced or at very low levels, were used to infect the murine macrophage-like cell line J774A.1. As predicted, within macrophages, cells of M. marinum containing pSAPM-GFP or pSAPC-GFP were brightly fluorescent, whereas the extracellular bacteria had minimal fluorescence (FIG. 5B). M. marinum strains containing the cloning vector pFPV27 (FIG. 5B) or the construct in which sapM promoter was inserted in the opposite orientation (data not shown) were nonfluorescent. This result, together with our in vitro data described above, indicates that sapM is induced by the mildly acidic environment of mycobacterial phagosome of cultured macrophages.

The sapM Gene is Present in Pathogenic Mycobacteria Only

As described above, SapM protein and enzyme activity were not detected in M. smegmatis, a nonpathogenic Mycobacterium species. To examine if SapM is specific to pathogenic mycobacteria, we analyzed two other Mycobacterium species, the nonpathogenic Mycobacterium chelonae and the fish pathogen, M. marinum. Like M. bovis BCG, the CFP of M. marinum culture grown in ammonium-containing Sauton medium exhibited a high level of acid phosphatase activity (data not shown), and this activity was dependent on the pH of the culture media (data not shown). In contrast, M. chelonae was similar to M. smegmatis and exhibited a residual level of acid phosphatase activity that was not regulated by media pH (data not shown). These results suggest that M. marinum contains a SapM homolog, whereas M. chelonae and M. smegmatis do not. Searching the unpublished genome sequences available at TIGR and the Sanger center revealed that SapM homologs are present in M. marinum and Mycobacterium avium (a bird pathogen), but not M. smegmatis (FIG. 6A).

To confirm the above results, DNA hybridizations of chromosomal DNA of M. smegmatis and M. chelonae using a radiolabeled probe specific for M. tuberculosis sapM were performed. Chromosomal DNA of M. bovis BCG and the sapM-containing plasmid pSAP were included as positive controls. The result showed that the sapM allele was not detected in M. smegmatis and M. chelonae chromosomes (FIG. 6B).

Immunogenicity of SapM

The antigenicity of SapM in human infection was assessed by Western blot using partially purified M. tuberculosis SapM and sera collected from seven individuals. These individuals included one patient diagnosed with lymph node TB who began chemotherapy four months prior to collection of the serum; one patient who was sputum smear-positive for M. avium; two individuals who had been vaccinated with M. bovis BCG; three apparently healthy individuals including one involved with patient care and one who tested PPD positive. Interestingly, sera from two patients (TB patient and patient infected by M. avium) reacted with SapM (lanes 4 and 5, FIG. 7), whereas sera from two healthy individuals including the one tested for PPD-positive did not react with SapM (lanes 2 and 6). However, sera from two individuals who had been vaccinated with BCG (lanes 7 and 8) and the individual involved in TB patient care (lane 3) also reacted with SapM. These results suggest that SapM is recognized by antibodies from TB patients, or from individuals who had been vaccinated with BCG, but not from healthy individuals who had not been exposed to M. tuberculosis.

Mycobacterium tuberculosis produces a secreted acid phosphatase, SapM, which exhibits remarkably high activity towards GTP and NADPH. The biological functions of SapM are unknown. In this study, we found that sapM is regulated by pH. The expression level and enzyme activity of SapM are dramatically increased (up to ˜30 fold) when the pH of culture medium is lowered from 7.0-7.4 to 5.8-6.6. Transcriptional fusions of truncated sapM with the promoterless gfp demonstrated that sapM expression is induced in infected macrophages. DNA hybridizations and sequence analyses indicated that the sapM gene is present in pathogenic mycobacteria including Mycobacterium bovis BCG, Mycobacterium avium, and Mycobacterium marinum, but is absent from nonpathogenic mycobacteria such as Mycobacterium smegmatis and Mycobacterium chelonae. Moreover, antibodies from TB patients or BCG vaccinated individuals but not from healthy individuals recognize SapM. Collectively, these results suggest that SapM is important for mycobacterial pathogenesis, which may contribute to intracellular survival by interfering with effector molecules involved in phagosome maturation.

Materials and Methods

Strains and Culture Conditions

M. bovis BCG strains, BCG-Japan, -Pasteur, -Frappier, and -Birkhaug, were provided by Marcel Behr (McGill University). M. marinum 1218R strain was provided by Lucia Barker (Rocky Mountain Laboratories, NIAID). M. smegmatis mc²-155 and M. chelonae PS4770 were described previously (Liu et al., 1995; Liu et al., 1996).

Mycobacterial cells were routinely grown in normal Sauton media containing (per liter): 0.5 g KH₂PO₄, 0.14 g MgSO₄, 2.0 g citric acid, 0.05 g ferric ammonium citrate, 5.0 g asparagine, and 60 ml glycerol. The pH was adjusted to 7.4. To study the effect of phosphate concentration on acid phosphatase activity, the bacteria were grown in Sauton medium to exponential phase, washed with the same medium without KH₂PO₄ and inoculated in 100 ml of Sauton medium in which the phosphate concentration was modified as follows (per liter): KH₂PO₄ was increased by 10 fold to 5.0 g (high Pi medium), or reduced by 10 fold to 0.05 g (low Pi medium). For studying the effect of nitrogen source on acid phosphatase, asparagine (5 g/l) in the original Sauton was substituted with NH₄Cl (1.42 g/l). For carbon source starvation, glycerol was omitted from the Sauton medium. For studying the effect of pH on acid phosphatase activity, the pH of the original Sauton medium was adjusted with buffers: 20 mM MOPS (for pH 7.0 and 6.6), or 20 mM MES (for pH 6.2 and 5.8). All cultures were grown with continuous shaking at 37° C., except for M. marinum and M. chelonae, which were grown at 30° C.

Molecular Cloning

An ordered BAC library of M. tuberculosis H37Rv genome was used as DNA template for cloning. Standard protocols were used for manipulation of DNA. Cloning of sapM was accomplished by ligating a 3 kb NheI fragment of BAC403, containing Rv3309 (upp), Rv331 0 (sapM) and Rv3311 (sapc), into the unique XbaI site of plasmid pMD31 to generate pSAP. Transcriptional fusions to a promoterless mutant gfp were constructed by cloning fragments of pSAP into pFPV27. To construct pSAPC-GFP, a 2.56 kb EcoRV fragment of pSAPM, comprising 560 bp upstream of sapM, the complete sapM gene, and sapC (Rv3311) truncated 183 bp from its 3′ end, was inserted into the unique EcoRV site of pFPV27 (Barker et al., 1998). To generate pSAPM-GFP, vector pSAPC-GFP was cut with BglII and the 3.8 kb and 2.5 kb fragments, comprising pFPV27, 560 bp upstream of the sapM start codon and all but 67 bp of the sapM coding region, were re-ligated. Plasmids were introduced into M. marinum and M. smegmatis mc²-155 by electroporation, and recombinants were selected on Middlebrook 7H9 agar (Difco) supplemented with 10% OADC enrichment and 25 μg/ml kanamycin.

Southern Blotting

Southern blotting was performed according to standard methods. Briefly, chromosomal DNA was isolated from M. bovis BCG, M. smegmatis mc²-155, and M chelonae, digested with EcoRI or EcoRV, separated on an agarose gel and transferred to a nylon membrane (Hybond N+, Amersham Pharmacia). Membranes were then hybridized with α³²P-CTP radiolabeled sapM probe (a 660 bp HphI-NdeI fragment of pSAP) and exposed on a phosphoimager cassette.

Enzyme Assays

For assay of secreted acid phosphatase activity, the culture filtrate protein (CFP) fractions were collected from cultures grown to exponential phase (A₆₀₀ ˜0.8), concentrated by centrifugal filters (Millipore), and dialyzed (150-200×volumes) against distilled water overnight at 4° C. Protein concentrations were determined using the BCA assay (Pierce). Acid phosphatase assay was carried out using p-nitrophenylphosphate (pNPP) at pH 6.8 in a microtitre format as described previously (Saleh and Belisle, 2000). Specific activity was calculated using an extinction coefficient for nitrophenol of 18,380 mM hr⁻¹ cm⁻¹, and the phosphatase activity was expressed as nmol hr⁻¹ mg⁻¹ (total CFP protein).

For assay of cell surface-associated acid phosphatase and alkaline phosphatase, bacterial cells were collected and briefly sonicated to disrupt cell clumps. Cells were then washed in Tris-buffered saline three times followed by a final wash in distilled water and cell density was adjusted to a final OD₅₉₅ of 0.42. The phosphatase activity of whole cells toward pNPP was assayed by measuring p-nitrophenol released as described above. Typically, 50 μl of bacterial cells were mixed with 150 μl of 25 mM acetate buffer, pH 6.0 (for acid phosphatase assay) or 25 mM Tris, pH 10.0 (for alkaline phosphatase). Reaction buffers also contained 1 mM each of MgCl₂ and ZnCl₂. The reactions were initiated by the addition of pNPP to a final concentration of 1 mg/ml. Following incubation at 37° C. for 30 min., cells were pelleted by centrifugation and the top 150 μl of the reactions were transferred to ELISA plate wells containing 50 μl of 1.0 M NaOH, which stopped the reaction, and the OD_{405 nm} measured in an ELISA reader.

Generation of Anti-SapM Antisera

A peptide, corresponding to residues 201-208 in the SapM amino acid sequence, was synthesized using solid phase peptide synthesis. The peptide was modified by acetylation of the carboxyl end and by the addition of a cysteine at the amino end. Approximately 10 mg of the purified peptide, having the sequence CNDGHDGSI-Ac, was reduced by dissolving in 4.0 ml of 10 mM ammonium bicarbonate buffer, pH 8.0 containing 20 mM DTT (buffer A) and incubating at room temperature for 2 hours, The reduced peptide was loaded onto an 1.5 cm×4.0 cm anion-exchange column (DEAE-Sephadex A50, Pharmacia) pre-equilibrated in buffer A, followed by washing with 25 ml of same buffer without DTT. Bound peptide was eluted with 10 ml of 10 mM acetate buffer, pH 3.0. The peptide, in a total volume of 2.0 ml, was immediately added to a tube containing 2 mg of mcKLH (Pierce Sciences), followed by the addition of 200 μl of 1.0 M phosphate buffer, pH 7.4 and mixed continuously at room temperature for 2 hours. The conjugated KLH was subsequently desalted on a G-10 column (1.5 cm×25 cm) in PBS. Two New Zealand rabbits were primed with 250 μg of the KLH conjugate, followed by a first boost at 28 days and a second boost at 56 days (100 μg each). Sera were screened 9 days following the second boost and the antisera collected the following day.

SDS-Polyacrylamide Gel Electrophoresis and Western Blotting

For detection of SapM protein, CFP samples (typically 8-10 μg protein) were separated on sodium dodecyl sulfate (SDS)-14% polyacrylamide gels, and visualized by silver staining, or for Western blotting, transferred to nitrocellulose membranes. The membranes were then probed with a 1:400 dilution of anti-SapM antisera and developed with anti-rabbit IgG-alkaline phosphatase conjugate and BCIP/NBT. For assay of SapM antigenicity, SapM protein was partially purified from the recombinant M. smegmatis strain carrying the plasmid pSAP, using methods described previously (Saleh and Belisle, 2000). The protein was run on 14% SDS-PAGE and transferred to nitrocellulose membranes. Individual lanes were cut and probed with a 1:150 dilution of individual human sera and developed as described above. Human serum samples were provided by Tony Mazzulli (Mount Sinai Hospital, Toronto, ON).

Fluorescence Microscopy

The mouse macrophage cell line J774 was maintained in RPMI 1640 media containing fetal bovine serum (5%), 100 μg/ml streptomycin, and 100 units/ml penicillin. For infections with M. marinum, cells were allowed to adhere to glass cover slips (22×22 mm) overnight in incubator at 37° C., 5% CO₂. Spent media was then replaced with fresh media (without antibiotics) and M. marinum was added at MOI of 10:1 and incubated at 37° C. for 1 hr. Excess bacilli were removed by several washes with sterile PBS and the infected cells were finally covered with fresh media and returned to the incubator. At given times post infection, the cover slips were washed several times with PBS, fixed for 15 minutes with 3.7% paraformaldehyde, permeabilized for 10 minutes with 0.1% Triton X-100, and stained with Texas-red phalloidin for 1 hr. All treatments were at room temperature. Images were collected on a Leica DM IRBE microscope equipped with a Hamamatsu digital camera. Images were collected using differential interference contrast (DIC), with a FITC filter to visualize GFP, or with a Texas red filter to visualize macrophages treated with cytoskeleton-specific stain pholloidin conjugated with Texas red.

The present invention has been described in detail and with particular reference to the preferred embodiments; however, it will be understood by one having ordinary skill in the art that changes can be made without departing from the spirit and scope thereof. For example, where the application refers to proteins, it is clear that peptides and polypeptides may often be used. Likewise, where a gene is described in the application, it is clear that nucleic acids or gene fragments may often be used.

All publications (including Genbank entries), patents and patent applications are incorporated by reference in their entirety to the same extent as if each individual publication, patent or patent application was specifically and individually indicated to be incorporated by reference in its entirety.

### REFERENCES

Barker, L. P., Brooks, D. M., and Small, P. L. (1998) The identification of Mycobacterium marinum genes differentially expressed in macrophage phagosomes using promoter fusions to green fluorescent protein. Mol Microbiol 29: 1167-1177.
Berthet, F. X., Lagranderie, M., Gounon, P., Laurent, W., Ensergueix, D., Chavarot, P., Thouron, F., Maranghi, E., Pelicic, V., Portnoi, D., Marchal, G., and Gicquel, B. (1998) Attenuation of virulence by disruption of the Mycobacterium tuberculosis erp gene. Science 282: 759-762.
Betts, J. C., Lukey, P. T., Robb, L. C., McAdam, R. A., and Duncan, K. (2002) Evaluation of a nutrient starvation model of Mycobacterium tuberculosis persistence by gene and protein expression profiling. Mol Microbiol 43: 717-731.
Braibant, M., and Content, J. (2001) The cell surface associated phosphatase activity of Mycobacterium bovis BCG is not regulated by environmental inorganic phosphate. FEMS Microbiol Left 195: 121-126.
Clemens, D. L., and Horwitz, M. A. (1995) Characterization of the Mycobacterium tuberculosis phagosome and evidence that phagosomal maturation is inhibited. J Exp Med 181: 257-270.
Clemens, D. L., Lee, B. Y., and Horwitz, M. A. (2000) Deviant expression of Rab5 on phagosomes containing the intracellular pathogens Mycobacterium tuberculosis and Legionella pneumophila is associated with altered phagosomal fate. Infect Immun 68: 2671-2684.
Cole, S. T.;, Brosch, R., Parkhill, J., Gamier, T., Churcher, C., Harris, D., Gordon, S. V., Eiglmeier, K., Gas, S., Barry, C. E., Tekaia, F., Badcock, K., Basham, D., Brown, D., Chillingworth, T., Connor, R., Davies, R., Devlin, K., Feltwell, T., Gentles, S., Hamlin, N., Holroyd, S., Hornsby, T., Jagels, K., and Barrell, B. G. (1998) Deciphering the biology of Mycobacterium tuberculosis from the complete genome sequence. Nature 393: 537-544.
Dowling, J. N., Saha, A. K., and Glew, R. H. (1992) -Virulence factors of the family Legionellaceae. Microbiol Rev 56: 32-60.
Fisher, M. A., Plikaytis, B. B., and Shinnick, T. M. (2002) Microarray analysis of the Mycobacterium tuberculosis transcriptional response to the acidic conditions found in phagosomes. J Bacteriol 184: 4025-4032.
Kuehnel, M. P., Goethe, R., Habermann, A., Mueller, E., Rohde, M., Griffiths, G., and Valentin, W. (2001) Characterization of the intracellular survival of Mycobacterium avium ssp. paratuberculosis: phagosomal pH and fusogenicity in J774 macrophages compared with other mycobacteria. Cell Microbiol 3: 551-566.
Liu, J., Rosenberg, E. Y., and Nikaido, H. (1995) Fluidity of the lipid domain of cell wall from Mycobacterium chelonae. Proc Natl Acad Sci USA 92: 11254-11258.
Liu, J., Takiff, H. E., and Nikaido, H. (1996) Active efflux of fluoroquinolones in Mycobacterium smegmatis mediated by LfrA, a multidrug efflux pump. J Bacteriol 178: 3791-3795.
Lucas, R. L., and Lee, C. A. (2000) Unraveling the mysteries of virulence gene regulation in Salmonella typhimurium. Mol Microbiol 36: 1024-1033.
McKinney, J. D., Honer, Z., Munoz, E., Miczak, A., Chen, B., Chan, W. T., Swenson, D., Sacchettini, J. C., Jacobs, W. R., and Russell, D. G. (2000) Persistence of Mycobacterium tuberculosis in macrophages and mice requires the glyoxylate shunt enzyme isocitrate lyase. Nature 406: 735-738.
Reilly, T. J., Baron, G. S., Nano, F. E., and Kuhlenschmidt, M. S. (1996) Characterization and sequencing of a respiratory burst-inhibiting acid phosphatase from Francisella tularensis. J Biol Chem 271: 10973-10983.
Rossolini, G. M., Schippa, S., Riccio, M. L., Berlutti, F., Macaskie, L. E., and Thaller, M. C. (1998) Bacterial nonspecific acid phosphohydrolases: physiology, evolution and use as tools in microbial biotechnology. Cell Mol Life Sci 54: 833-850.
Saleh, M. T., and Belisle, J. T. (2000) Secretion of an acid phosphatase (SapM) by Mycobacterium tuberculosis that is similar to eukaryotic acid phosphatases. J Bacteriol 182: 6850-6853.
Sturgill, K., Schlesinger, P. H., Chakraborty, P., Haddix, P. L., Collins, H. L., Fok, A. K., Allen, R. D., Gluck, S. L., Heuser, J., and Russell, D. G. (1994) Lack of acidification in Mycobacterium phagosomes produced by exclusion of the vesicular proton-ATPase. Science 263: 678-681.
Takai, Y., Sasaki, T., and Matozaki, T. (2001) Small GTP-binding proteins. Physiol Rev 81: 153-208.
Teitelbaum, R., Cammer, M., Maitland, M. L., Freitag, N. E., Condeelis, J., and Bloom, B. R. (1999) Mycobacterial infection of macrophages results in membrane-permeable phagosomes. Proc Natl Acad Sci USA 96: 15190-15195.
Triccas, J. A., and Gicquel, B. (2000) Life on the inside: probing Mycobacterium tuberculosis gene expression during infection. Immunol Cell Biol 78: 311-317.
Via, L. E., Deretic, D., Ulmer, R. J., Hibler, N. S., Huber, L. A., and Deretic, V. (1997) Arrest of mycobacterial phagosome maturation is caused by a block in vesicle fusion between stages controlled by rab5 and rab7. J Biol Chem 272: 13326-13331.
Westhoff, C. M., Ferreri, J., Mak, D. O., and Foskett, J. K. (2002) Identification of the erythrocyte Rh blood group glycoprotein as a mammalian ammonium transporter. J Biol Chem 277-:12499-12502.
Xu, S., Cooper, A., Sturgill, K., van, H., Chatterjee, D., Orme, I., Allen, P., and Russell, D. G. (1994) Intracellular trafficking in Mycobacterium tuberculosis and Mycobacterium avium-infected macrophages. J Immunol 153: 2568-2578.
Yuan, Y., Crane, D. D., Simpson, R. M., Zhu, Y. Q., Hickey, M. J., Sherman, D. R., and Barry, C. E. (1998) The 16-kDa alpha-crystallin (Acr) protein of Mycobacterium tuberculosis is required for growth in macrophages. Proc Natl Acad Sci USA 95:9578-9583.

1. An isolated DNA sequence comprising a promoter or a promoter fragment of a mycobacterial secreted acid phosphatase gene wherein the promoter or the promoter fragment is sufficient to control expression of a nucleotide sequence of interest and is inducible under low-pH conditions.

2. The isolated DNA sequence of claim 1, wherein the promoter or the promoter fragment is selected from the group consisting of a Mycobacterium tuberculosis sapM promoter or promoter fragment, a Mycobacterium bovis sapM promoter or promoter fragment, a Mycobacterium avium sapM promoter or promoter fragment, and a Mycobacterium marinum sapM promoter or promoter fragment.

3. An isolated DNA sequence comprising a promoter or a promoter fragment sufficient to control expression of a nucleotide sequence of interest and inducible under low-pH conditions, wherein the promoter or the promoter fragment hybridize to a sapM promoter selected from the group consisting of a Mycobacterium tuberculosis sapM promoter [SEQ ID NO:1], a Mycobacterium bovis sapM promoter [SEQ ID NO:2], a Mycobacterium avium sapM promoter [SEQ ID NO:3] and a Mycobacterium marinum sapM promoter [SEQ ID NO:4] under high stringency hybridization conditions.

4. An expression vector comprising the isolated DNA sequence of claim 1.

5. A host cell transformed with the vector of claim 4.

6. A transcription cassette comprising:
(a) a mycobacterial secreted acid phosphatase promoter or promoter fragment, wherein the promoter or the promoter fragment is sufficient to control expression of a nucleotide sequence of interest;
(b) a nucleotide sequence of interest operably linked to the promoter or the promoter fragment; and
(c) a transcriptional termination region.

7. The transcription cassette of claim 6 wherein the mycobacterial secreted acid phosphatase promoter or promoter fragment is selected from the group consisting of a Mycobacterium tuberculosis sapM promoter [SEQ ID NO:1], a Mycobacterium bovis sapM promoter [SEQ ID NO:2], a Mycobacterium avium sapM promoter [SEQ ID NO:3], and a Mycobacterium marinum sapM promoter [SEQ ID NO:4].

8. The transcription cassette of claim 6 further comprising a mycobacterial secreted acid phosphatase N-terminal signal sequence.

9. The transcription cassette of claim 8 wherein the mycobacterial secreted acid phosphatase N-terminal signal sequence is selected from the group consisting of a Mycobacterium tuberculosis sapM N-terminal signal sequence [SEQ ID NO:5], a Mycobacterium bovis sapM N-terminal signal sequence [SEQ ID NO:6], a Mycobacterium avium sapM N-terminal signal sequence [SEQ ID NO:7], and a Mycobacterium marinum sapM N-terminal signal sequence [SEQ ID NO:8].

10.-27. (canceled)

28. A vaccine or immunogenic composition for treatment or prophylaxis of a mammal against challenge by a mycobacterium comprising the isolated DNA sequence claim 1.

29.-30. (canceled)

31. An expression vector comprising the isolated DNA sequence of claim 2.

32. An expression vector comprising the isolated DNA sequence of claim 3.

33. A host cell transformed with the vector of claim 31.

34. A host cell transformed with the vector of claim 32.

35. The transcription cassette of claim 7 further comprising a mycobacterial secreted acid phosphatase N-terminal signal sequence.

36. The transcription cassette of claim 35 wherein the mycobacterial secreted acid phosphatase N-terminal signal sequence is selected from the group consisting of a Mycobacterium tuberculosis sapM N-terminal signal sequence [SEQ ID NO:5], a Mycobacterium bovis sapM N-terminal signal sequence [SEQ ID NO:6], a Mycobacterium avium sapM N-terminal signal sequence [SEQ ID NO:7], and a Mycobacterium marinum sapM N-terminal signal sequence [SEQ ID NO:8].

37. A vaccine or immunogenic composition for treatment or prophylaxis of a mammal against challenge by a mycobacterium comprising the isolated DNA sequence claim 2.

38. A vaccine or immunogenic composition for treatment or prophylaxis of a mammal against challenge by a mycobacterium comprising the isolated DNA sequence claim 3.
